(19) United States
(12) Patent Application Publication
(10) Pub. No.: US 2025/0366928 A1
(43) Pub. Date: Dec. 4, 2025

(54) Mount Assemblies With Anchors For Use With Navigated Surgical Systems

(71) Applicant: Mobius Imaging, LLC, Shirley, MA (US)

(72) Inventors: Amir Ali Sharifi-Mehr, Bloomingdale, NJ (US); Robert Waldrop, Scottsdale, AZ (US); Michael Y. Wang, North Miami, FL (US); Kornelis Poelstra, Las Vegas, NV (US)

(73) Assignee: Mobius Imaging, LLC, Shirley, MA (US)

(21) Appl. No.: 18/876,748

(22) PCT Filed: Jun. 29, 2023
(86) PCT No.: PCT/US2023/026540

§ 371 Date: Dec. 19, 2024

Related U.S. Application Data
(60) Provisional application No. 63/356,606, filed on Jun. 29, 2022.

(51) Int. Cl. A61B 34/20; A61B 90/00; A61B 90/50

(52) U.S. Cl. CPC A61B 34/20; A61B 90/08; A61B 90/39; A61B 90/50; A61B 2034/2068

(57) ABSTRACT

A mount assembly for use with a navigable tracker. The mount assembly includes a frame, a coupler for releasably securing the tracker, and an anchor extending along an axis between a distal end for engaging tissue and a proximal end arranged to receive impaction force. The anchor includes an arrow body coupled to a shank and having a tip tapering towards the distal end for advancing into engagement with tissue, and a pair of wing braces to inhibit rotation of the anchor relative to engaged tissue. A guide operatively attached to the frame defines a bore to receive the shank, and a guide lock is operable between: a released configuration to permit movement of the shank along the bore, and a locked configuration to restrict movement of the shank along the bore to effect concurrent movement of the tracker with the tissue engaged by the anchor.

### CROSS-REFERENCE TO RELATED APPLICATION

The subject patent application claims priority to and all the benefits of U.S. Provisional Patent Application No. 63/356,606 filed on Jun. 29, 2022, the disclosure of which is hereby incorporated by reference in its entirety.

### BACKGROUND

Navigation systems are frequently utilized to assist medical professionals in carrying out various types of surgical procedures, including neurosurgical and orthopedic procedures. To this end, a surgeon may utilize a navigation system to track, monitor, or otherwise locate one or more tools, surgical instruments, and/or portions of a patient's anatomy within a common reference frame. Typically, tools and/or surgical instruments are tracked together with the anatomy, and their relative movement is depicted on a display.

Conventional navigation systems may employ light signals, sound waves, magnetic fields, radio frequency signals, and the like, in order to track the position and/or orientation of objects. Often, trackers are attached or otherwise integrated into the object being tracked. A localizer cooperates with tracking elements (e.g., fiducials, markers, and the like) coupled to the fixation tool to monitor the fixation tool, and ultimately to determine a position and/or orientation of the object being tracked.

For certain procedures, patient-specific imaging data may be acquired intraoperatively using one or more types of imaging systems to help assist the surgeon in visualizing, navigating relative to, and/or treating the anatomy. To this end, navigation systems may cooperate with imaging systems and/or other parts of surgical systems (e.g., surgical tools, instruments, surgical robots, and the like) to track objects relative to a target site of the anatomy.

In certain surgical procedures, such as orthopedic procedures involving the correction, stabilization, resection, or replacement of one or more parts of a patient's body, such as to help improve patient mobility, reduce pain, mitigate the risk of subsequent injury or damage, and the like, a trackers may be secured to various portions of the anatomy.

Depending on the type of surgical procedure being performed, the location and arrangement of the target site, and/or the specific configuration of the navigation system, it may be advantageous to secure trackers to tissue at or otherwise adjacent to the target site prior to acquiring patient-specific imaging data via the imaging system (e.g., to facilitate registration of the imaging data with the navigation system). In such circumstances, when securing the tracker to the anatomy, the surgeon generally considers the visibility of the tracker to the navigation system, the arrangement of the tracker relative to the target site, and/or the arrangement of the tracker relative to the intended position(s) of the imaging system.

While certain types of trackers generally remain fixed relative to tissue when anchored, other types of trackers may be adjustably positioned or articulated relative to the tissue after attachment. However, conventional adjustable trackers may employ relatively large adjustable linkages, which may employ joints that are individually articulable relative to each other to help facilitate adjustable positioning of the tracker in multiple degrees of freedom. However, it will be appreciated that each of these joints needs to remain secure in order to ensure that the tracker can be accurately monitored. Furthermore, it will be appreciated that these types of linkages tend to result in the tracker being relatively bulky, and may present an increased risk of inadvertently obscuring or limiting access to the target site from certain approaches, and/or an increased risk of “bumping” or jostling the tracker (e.g., with a tool, with a portion of the imaging device, and the like) and leading to tracking inaccuracies. Here too, for trackers which utilize a single threaded anchor to remain fixed relative to tissue when inserted, maintaining torsional stability can be difficult to reliably achieve.

Therefore, while navigation systems that use threaded anchors have generally worked well for their intended purpose, there remains a need in the art to overcome one or more of the deficiencies described above.

### SUMMARY

The present disclosure provides a mount assembly for use with a navigable tracker, the mount assembly may include: a frame; a coupler operatively attached to the frame for releasably securing the tracker; an anchor extending along an axis between a distal end for engaging tissue and a proximal end arranged to receive impaction force, the anchor including: a shank disposed along the axis between the distal end and the proximal end, an arrow body coupled to the shank and having a tip tapering towards the distal end for advancing into engagement with tissue with a pair of wing braces extending away from the axis to inhibit rotation of the anchor about the axis relative to engaged tissue; a guide operatively attached to the frame and defining a bore shaped to receive the shank of the anchor; and a guide lock operable between: a released configuration to permit movement of the shank along the bore, and a locked configuration to restrict movement of the shank along the bore to effect concurrent movement of the tracker with the tissue engaged by the anchor.

The present disclosure also provides a mount assembly for use with a navigable tracker, the mount assembly may include: a frame defining a coupler seat; a coupler including a perch disposed in the coupler seat and arranged for selective movement relative to the frame about a coupler point, and a tracker interface spaced from the perch for releasably securing the tracker; a coupler lock operatively attached to the frame and selectively operable between: a secured configuration to restrict movement of the coupler relative to the frame, and a movable configuration to permit limited movement of the coupler relative to the frame about the coupler point; an anchor extending along an axis between a distal end for engaging tissue and a proximal end arranged to receive impaction force, the anchor including: a shank disposed along the axis between the distal end and the proximal end, an arrow body coupled to the shank and having a tip tapering towards the distal end for advancing into engagement with tissue with a pair of wing braces extending away from the axis to inhibit rotation of the anchor about the axis relative to engaged tissue; a guide operatively attached to the frame and defining a bore shaped to receive the shank of the anchor; and a guide lock operable between: a released configuration to permit movement of the shank along the bore, and a locked configuration to restrict movement of the shank along the bore to effect concurrent movement of the tracker with the tissue engaged by the anchor.

### BRIEF DESCRIPTION OF THE DRAWINGS

FIG. 1 is a perspective view of a surgical system shown comprising a navigation system and an imaging system supporting a patient with tracker assemblies secured adjacent to a target site according to versions of the present disclosure.

FIG. 2 is a partial perspective view of the tracker assemblies of FIG. 1 secured adjacent to the target site of the patient, the tracker assemblies each shown having a navigable tracker secured to a respective mount assembly.

FIG. 3A is a perspective view of one of tracker assemblies of FIGS. 1-2, the mount assembly shown having a frame subassembly with a coupler supporting the tracker in a first exemplary tracker arrangement relative to a frame via a coupler lock, and an anchor disposed in the bore supported by the frame and locked in the first exemplary anchor arrangement relative to the frame via guide locks.

FIG. 3B is another perspective view of the tracker assembly of FIG. 3B, shown with the coupler of the mount assembly supporting the tracker in a second exemplary tracker arrangement relative to the frame, and with the anchor locked in the second exemplary anchor arrangement relative to the frame.

FIG. 4A is a perspective view of the anchor of FIGS. 3A-3B shown arranged adjacent to a target site of a patient's anatomy.

FIG. 4B is another perspective view of the anchor of FIG. 4A shown with an impaction receiver arranged adjacent to a proximal end of the anchor.

FIG. 4C is another perspective view of the anchor and the impaction receiver of FIG. 4B, shown with the impaction receiver positioned at the proximal end of the anchor prior to impaction at the target site.

FIG. 4D is another perspective view of the anchor and the impaction receiver of FIG. 4C, shown with the anchor and the impaction receiver advanced towards the target site following impaction.

FIG. 4E is another perspective view of the anchor of FIGS. 4A-4D secured to the target site following impaction as depicted in FIG. 4D, and shown with the frame assembly of the tracker assembly of FIGS. 2-3B arranged adjacent to the proximal end of the anchor.

FIG. 4F is another perspective view of the anchor secured as depicted in FIG. 4E, and shown with the anchor accepting engagement from the frame assembly of the tracker assembly of FIGS. 2-3B arranged along an axis of a shank.

FIG. 4G is a partially exploded perspective view of the tracker assembly of FIGS. 4E-4F shown with a tracker spaced from a coupler of the tracker assembly and a marker spaced from the impaction end of the anchor.

FIG. 4H is a perspective view of the tracker assembly of FIGS. 2-4E, shown locked in the first exemplary anchor arrangement relative to the frame via guide locks while advanced in the patient.

FIG. 4I is another perspective view of the tracker assembly of FIGS. 2-4E, shown locked in the second exemplary anchor arrangement relative to the frame via guide locks while advanced in the patient.

FIG. 5 is a partially exploded perspective view of the frame assembly of FIGS. 4E-4I, shown with a second version with an anchor spaced from the frame assembly and from the first version of the anchor of FIGS. 3A-4I.

FIG. 6A is a sectional view of the first version of the anchor taken along line 6A-6A in FIG. 5.

FIG. 6B is a sectional view of the second version of the anchor taken along line 6B-6B in FIG. 5.

FIG. 7A is a partial perspective view of a portion of the anchor of FIGS. 3A-5.

FIG. 7B is a partial perspective view of a portion of another version of the anchor depicted in FIG. 7A.

FIG. 8 is a broken, right-side plan view of the frame assembly of FIGS. 2-4I.

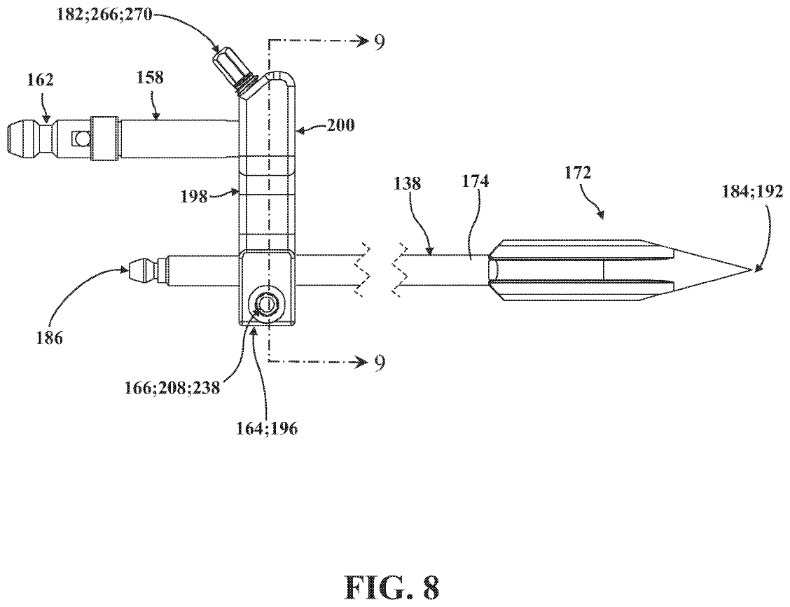

FIG. 9A is a section view of the mount assembly taken along line 9-9 in FIG. 8, shown with the guide lock arranged in a locked configuration to restrict movement of the anchor relative to the frame.

FIG. 9B is another section view of the mount assembly of FIG. 9A, shown with the guide lock arranged in a released configuration to simultaneously permit pivoting and translational movement of the anchor relative to the frame.

FIG. 10A is an exploded perspective view of the frame subassembly of the mount assembly of FIGS. 2-5.

FIG. 10B is another exploded perspective view of the frame subassembly of the mount assembly of FIGS. 2-5.

FIG. 11 is a top-side plan view of the frame subassembly of FIG. 10A.

FIG. 12A is a section view of the frame assembly taken along line 12-12 in FIG. 11, shown with the coupler lock arranged in a secured configuration to restrict movement of the coupler relative to the frame.

FIG. 12B is another section view of the mount assembly of FIG. 12A, shown with the coupler lock arranged in a movable configuration to permit limited movement of the coupler relative to the frame.

It will be appreciated that one or more of the versions depicted throughout the drawings may have certain components, structural features, and/or assemblies removed, depicted schematically, and/or shown in phantom for illustrative purposes.

### DETAILED DESCRIPTION

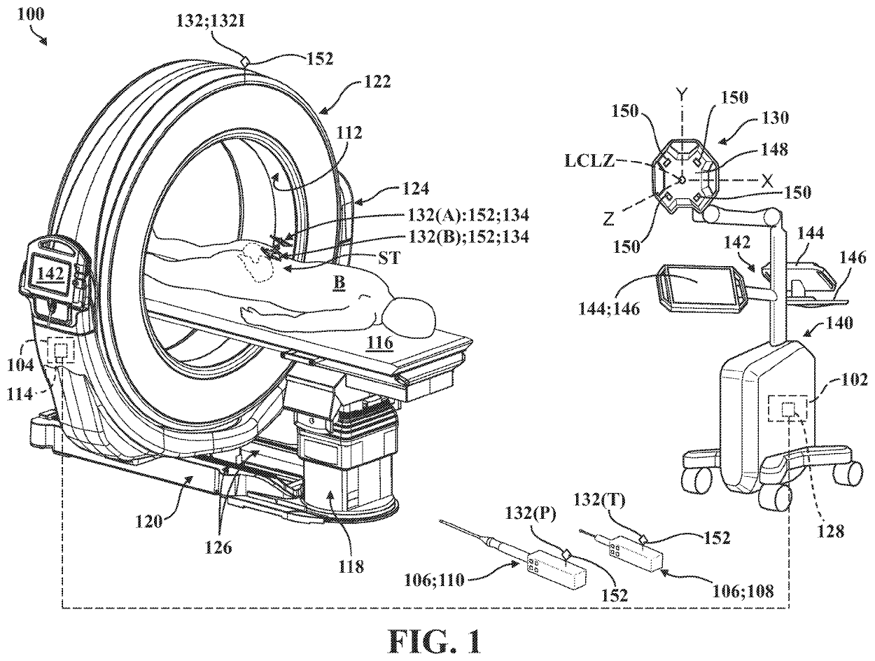

Referring now to the drawings, wherein like numerals indicate like or corresponding parts throughout the several views, a surgical system 100 is shown in FIG. 1 for treating a patient P. To this end, the illustrated surgical system 100 generally includes a navigation system 102, an imaging system 104, and one or more types of surgical instruments 106. As will be appreciated from the subsequent description below, the surgical system 100 is configured to, among other things, allow the surgeon to visualize, approach, and treat or otherwise manipulate anatomy of a patient P at a target site ST with a high level of control. To this end, imaging data ID of the target site ST may be acquired via the imaging system 104, and can be used to assist the surgeon in visualizing the patient's P anatomy at or otherwise adjacent to the target site ST. Here, the imaging data ID may also be utilized by the navigation system 102 to, among other things, facilitate navigation of surgical instruments 106 relative to the target site ST. Each of the components of the surgical system 100 introduced above will be described in greater detail below.

In FIG. 1, an operating room is shown with a patient P undergoing an exemplary surgical procedure performed using the surgical system 100. In this illustrative example, a minimally-invasive spinal surgical procedure, such as a posterior interbody spinal fusion, is being performed. It will be appreciated that this example is illustrative, and that other types of surgical procedures are contemplated. During the surgical procedure, one or more hand-held surgical instruments 106, such as a rotary tool 108 and/or a pointer tool 110, may be used by the surgeon. As noted above and as is described in greater detail below, the navigation system 102 may be configured to track states of one or more of the surgical instruments 106 relative to the target site ST. In this exemplary surgical procedure, the rotary tool 108 may be employed as a cutting or drilling tool to remove tissue T, form pilot holes (e.g., in the ilium, in vertebrae, and the like), or otherwise approach the target site ST. The rotary tool 108 may also be used to drive or otherwise install implantable components (e.g., pedicle screws, anchors, and the like).

For illustrative purposes, generically-depicted surgical instruments 106 configured for hand-held use are shown in FIG. 1. However, as will be appreciated from the subsequent description below, aspects of the surgical system 100 may be used with any suitable type of surgical instrument 106 without departing from the scope of the present disclosure. Furthermore, in addition to hand-held surgical instruments 106 of various types and configurations, aspects of the surgical system 100 may also be employed in connection with robotically-controlled surgical instruments 106 (not shown). Certain types of robotically-controlled surgical instruments 106 are disclosed in U.S. Pat. No. 9,119,655, entitled “Surgical Robotic arm Capable of Controlling a Surgical Instrument in Multiple Modes;” U.S. Pat. No. 10,456,207, entitled “Systems and Tools for use with Surgical Robotic Manipulators;” U.S. Patent Application Publication No. 2019/0231447, entitled “End Effectors And Methods For Driving Tools Guided By Surgical Robotic Systems;” U.S. Patent Application Publication No. 2016/0302871, entitled “Integrated Medical Imaging and Surgical Robotic System;” and U.S. Patent Application Publication No. 2020/0078097, entitled “Methods and Systems for Robot-Assisted Surgery,” the disclosures of each of which are hereby incorporated by reference in their entirety.

As noted above, the imaging system 104 may be used to obtain imaging tata ID of the patient, which may be a human or animal patient. In the representative version illustrated in FIG. 1, the imaging system 104 is realized as an x-ray computed tomography (CT) imaging device. Here, the patient P may be positioned within a central bore 112 of the imaging system 104 and an x-ray source and detector may be rotated around the central bore 112 to obtain raw x-ray imaging data ID of the patient P. The imaging data ID may be processed using an imaging system controller 114, or another suitable controller, in order to construct three-dimensional imaging data ID, two-dimensional imaging data ID, and the like, which may be transmitted to or otherwise utilized by the navigation system 102 or other components of the surgical system 100.

In some versions, imaging data ID may be obtained preoperatively (e.g., prior to performing a surgical procedure) or intraoperatively (e.g., during a surgical procedure) by positioning the patient P within the central bore 112 of the imaging system 104. In order to obtain imaging data ID, a portion of the imaging system 104 may be moved relative to a patient support 116 (e.g., a surgical table) on which the patient P is disposed while the patient P remains stationary. Here, the patient support 116 is secured to the imaging system 104, such as via a column 118 which is mounted to a base 120 of the imaging system 104. A portion of the imaging system 104 (e.g., an O-shaped imaging gantry 122) which includes at least one imaging component may be supported by an articulable support 124 that can translate along the length of the base 120 on rails 126 to perform an imaging scan of the patient P, and may translate away from the patient P to an out-of-the-way position for performing a surgical procedure on the patient P.

An example imaging system 104 that may be used in various versions is the AIRO® intra-operative CT system manufactured by Mobius Imaging, LLC. Examples of x-ray CT imaging devices that may be used according to various versions of the present disclosure are described in U.S. Pat. No. 10,151,810, entitled “Pivoting Multi-directional X-ray Imaging System with a Pair of Diametrically Opposite Vertical Support Columns Tandemly Movable Along a Stationary Base Support;” U.S. Pat. No. 9,962,132, entitled “Multi-directional X-ray Imaging System with Single Support Column;” U.S. Pat. No. 9,801,592, entitled “Caster System for Mobile Apparatus;” U.S. Pat. No. 9,111,379, entitled “Method and System for X-ray CT Imaging;” U.S. Pat. No. 8,118,488, entitled “Mobile Medical Imaging System and Methods;” and U.S. Patent Application Publication No. 2014/0275953, entitled “Mobile X-ray Imaging System,” the disclosures of each of which are hereby incorporated by reference in their entirety.

While the illustrated imaging system 104 is realized as an x-ray CT imaging device as noted above, in other versions, the imaging system 104 may comprise one or more of an x-ray fluoroscopic imaging device, a magnetic resonance (MR) imaging device, a positron emission tomography (PET) imaging device, a single-photon emission computed tomography (SPECT), or an ultrasound imaging device. Other configurations are contemplated. In some versions, the imaging system 104 may be a mobile CT device that is not attached to the patient support 116 and may be wheeled or otherwise moved over the patient P and the patient support 116 to perform a scan. Examples of mobile CT devices include the BodyTom® CT scanner from Samsung Electronics Co., Ltd. and the O-arm® surgical imaging system form Medtronic, plc. The imaging system 104 may also be a C-arm x-ray fluoroscopy device. In other versions, the imaging system 104 may be a fixed-bore imaging device, and the patient P may be moved into the bore of the device, either on a patient support 116 or on a separate patient table that is configured to slide in and out of the central bore 112. Further, although the imaging system 104 shown in FIG. 1 is located close to the patient P within the operating room, the imaging system 104 may be located remotely, such as in another room or building (e.g., in a hospital radiology department).

The surgical system 100 employs the navigation system 102 to, among other things, track movement of various objects, such as the surgical instruments 106 and parts of the patient's P anatomy (e.g., tissue at the surgical site ST), as well as portions of the imaging system 104 in some versions. To this end, the navigation system 102 comprises a navigation controller 128 coupled to a localizer 130 that is configured to sense the position and/or orientation of trackers 132 within a localizer coordinate system LCLZ. As is described in greater detail below, the trackers 132 (also referred to herein as “navigable trackers”) are fixed, secured, or otherwise attached to specific objects, and are configured to be monitored by the localizer 130.

The navigation controller 128 is disposed in communication with the localizer 130 and gathers position and/or orientation data for each tracker 132 sensed by the localizer 130 in the localizer coordinate system LCLZ. The navigation controller 128 may be disposed in communication with the imaging system controller 114 (e.g., to receive imaging data ID) and/or in communication with other components of the surgical system 100 (e.g., robotic arm controllers, tool controllers, and the like; not shown). However, other configurations are contemplated. The controllers 114, 128 may be realized as computers, processors, control units, and the like, and may be discrete components, may be integrated, and/or may otherwise share hardware.

It will be appreciated that the localizer 130 can sense the position and/or orientation of multiple trackers 132 to track correspondingly multiple objects within the localizer coordinate system LCLZ. By way of example, and as is depicted in FIG. 1, trackers 132 may comprise a tool tracker 132T, a pointer tracker 132P, an imaging system tracker 1321, a first patient tracker 132A, and/or a second patient tracker 132B, as well as additional patient trackers, trackers for additional medical and/or surgical tools, and the like.

In FIG. 1, the tool tracker 132T, the pointer tracker 132P, and the imaging system tracker 132I are each depicted generically and are shown firmly fixed to (or otherwise integrated with) the rotary tool 108, the pointer tool 110, and the gantry 122 of the imaging system 104, respectively. The patient trackers 132A, 132B, on the other hand, are removably coupled to mount assemblies 134 to define tracker assemblies 136 which facilitate selective movement of the trackers 132A, 132B relative to their mount assemblies 134 according to versions of the present disclosure, as described in greater detail below. Here, the tracker assemblies 136 are firmly fixed to different portions of the patient's P anatomy (e.g., to opposing lateral sides of the ilium) via an anchor 138 configured to releasably engage tissue (e.g., bone). It will be appreciated that trackers 132 may be firmly affixed to different types of tracked objects (e.g., discrete bones, tools, pointers, and the like) in a number of different ways.

The position of the patient trackers 132A, 132B relative to the anatomy of the patient P to which they are attached can be determined by known registration techniques, such as point-based registration in which pointer tool 110 (to which the pointer tracker 132P is fixed) is used to touch off on bony landmarks on bone, or to touch off on several points across the bone for surface-based registration. Conventional registration techniques can be employed to correlate the pose of the patient trackers 132A, 132B to the patient's anatomy. Other types of registration are also possible.

Position and/or orientation data may be gathered, determined, or otherwise handled by the navigation controller 128 using conventional registration/navigation techniques to determine coordinates of each tracker 132 within the localizer coordinate system LCLZ. These coordinates may be utilized by various components of the surgical system 100 (e.g., to facilitate control of the surgical instruments 106, to facilitate navigation based on imaging data ID, and the like).

In the representative version illustrated in FIG. 1, the navigation controller 128 and the localizer 130 are supported on a mobile cart 140 which is movable relative to the base 120 of the imaging system 104. The mobile cart 140 also supports a user interface, generally indicated at 142, to facilitate operation of the navigation system 102 by displaying information to, and/or by receiving information from, the surgeon or another user. The user interface 142 may be disposed in communication with other components of the surgical system 100 (e.g., with the imaging system 104), and may comprise one or more output devices 144 (e.g., monitors, indicators, display screens, and the like) to present information to the surgeon (e.g., images, video, data, a graphics, navigable menus, and the like), and one or more input devices 146 (e.g., buttons, touch screens, keyboards, mice, gesture or voice-based input devices, and the like).

In some versions, the surgical system 100 is capable of displaying a virtual representation of the relative positions and orientations of tracked objects to the surgeon or other users of the surgical system 100, such as with images and/or graphical representations of the anatomy of the patient P and the surgical instrument 106 presented on one or more output devices 144 (e.g., a display screen). The navigation controller 128 may also utilize the user interface 142 to display instructions or request information from the surgeon or other users of the surgical system 100. Other configurations are contemplated. One type of mobile cart 140 and user interface 142 of this type of navigation system 102 is described in U.S. Pat. No. 7,725,162, entitled “Surgery System,” the disclosure of which is hereby incorporated by reference in its entirety.

Because the mobile cart 140 and the gantry 122 of the imaging system 104 can be positioned relative to each other and also relative to the patient P in the representative version illustrated in FIG. 1, the navigation system 102 can transform the coordinates of each tracker 132 from the localizer coordinate system LCLZ into other coordinate systems (e.g., defined by different trackers 132, localizers 130, and the like), or vice versa, so that navigation relative to the target site ST (or control of surgical instruments 106) can be based at least partially on the relative positions and orientations of multiple trackers 132 within a common coordinate system (e.g., the localizer coordinate system LCLZ). Coordinates can be transformed using a number of different conventional coordinate system transformation techniques. It will be appreciated that the localizer 130 or other components of the navigation system 102 could be arranged, supported, or otherwise configured in other ways without departing from the scope of the present disclosure. By way of non-limiting example, the localizer 130 could be coupled to the imaging system 104 in some versions (e.g., to the gantry 122). Other configurations are contemplated.

In the illustrated version, the localizer 130 is an optical localizer and includes a camera unit 148 with one or more optical position sensors 150. The navigation system 102 employs the optical position sensors 150 of the camera unit 148 to sense the position and/or orientation of the trackers 132 within the localizer coordinate system LCLZ. To this end, the trackers 132 each employ one or more markers 152 (also referred to as “fiducials” in some versions) that are supported on an array 154 in a predetermined arrangement. However, as will be appreciated from the subsequent description below, trackers 132 may have different configurations, such as with different quantities of markers 152 that can be secured to or otherwise formed in other structures besides the arrays 154 illustrated throughout the drawings (e.g., various types of housings, frames, surfaces, and the like). Other configurations are contemplated.

In the representative version illustrated herein, the trackers 132 each employ “passive” markers 152 (e.g., reflective markers such as spheres, cones, and the like) which reflect emitted light that is sensed by the optical position sensors 150 of the camera unit 148. In some versions, trackers 132 could employ “active” markers 152 (e.g., light emitting diodes “LEDs”), which emit light that is sensed by the optical position sensors 150 of the camera unit 148. Examples of navigation systems 102 of these types are described in U.S. Pat. No. 9,008,757, entitled “Navigation System Including Optical and Non-Optical Sensors,” the disclosure of which is hereby incorporated by reference in its entirety. In some versions, the markers 152 may be provided with a coating formed from a radiopaque material such as barium, bismuth subcarbonate, barium sulfate, bismuth oxychloride, bismuth trioxide, tungsten and tantalum. This configuration can help promote visibility of the markers 152 in imaging data ID of the patient P in order to, among other things, facilitate registration, calibration, validation, and/or translation between reference frames and/or coordinate systems associated with different components of the surgical system 100.

Although one version of the mobile cart 140 and localizer 130 of the navigation system 102 is illustrated in FIG. 1, it will be appreciated that the navigation system 102 may have any other suitable configuration for monitoring trackers 132 which, as will be appreciated from the subsequent description below, may be of various types and configurations and could employ various types of markers 152. Thus, for the purposes of clarity and consistency, the term “marker 152” is used herein to refer to a portion of a tracker 132 (e.g., a passive marker 152 mounted to an array 154) that can be monitored by a localizer 130 to track (e.g., states, motion, position, orientation, and the like) of the object to which the tracker 132 is secured, irrespective of the specific type or configuration of the localizer 130 and/or tracker 132.

In some versions, the navigation system 102 and/or the localizer 130 could be radio frequency (RF) based. For example, the navigation system 102 may comprise an RF transceiver coupled to the navigation controller 128. Here, the trackers 132 may comprise markers 152 realized as RF emitters or transponders, which may be passive or may be actively energized. The RF transceiver transmits an RF tracking signal, and the RF emitters respond with RF signals such that tracked states are communicated to (or interpreted by) the navigation controller 128. The RF signals may be of any suitable frequency. The RF transceiver may be positioned at any suitable location to track the objects using RF signals effectively. Furthermore, it will be appreciated that versions of RF-based navigation systems may have structural configurations that are different than the navigation system 102 illustrated throughout the drawings.

In some versions, the navigation system 102 and/or localizer 130 may be electromagnetically (EM) based. For example, the navigation system 102 may comprise an EM transceiver coupled to the navigation controller 128. Here, the trackers 132 may comprise markers 152 realized as EM components (e.g., various types of magnetic trackers, electromagnetic trackers, inductive trackers, and the like), which may be passive or may be actively energized. The EM transceiver generates an EM field, and the EM components respond with EM signals such that tracked states are communicated to (or interpreted by) the navigation controller 128. The navigation controller 128 may analyze the received EM signals to associate relative states thereto. Here too, it will be appreciated that versions of EM-based navigation systems may have structural configurations that are different than the navigation system 102 illustrated throughout the drawings.

Those having ordinary skill in the art will appreciate that the navigation system 102 and/or localizer 130 may have any other suitable components or structure not specifically recited herein. Furthermore, any of the techniques, methods, and/or components described above with respect to the camera-based navigation system 102 shown throughout the drawings may be implemented or provided for any of the other versions of the navigation system 102 described herein. For example, the navigation system 102 may also be based on one or more of inertial tracking, ultrasonic tracking, image-based optical tracking (e.g., with markers 152 are defined by patterns, shapes, edges, and the like that can be monitored with a camera), or any combination of tracking techniques. Other configurations are contemplated.

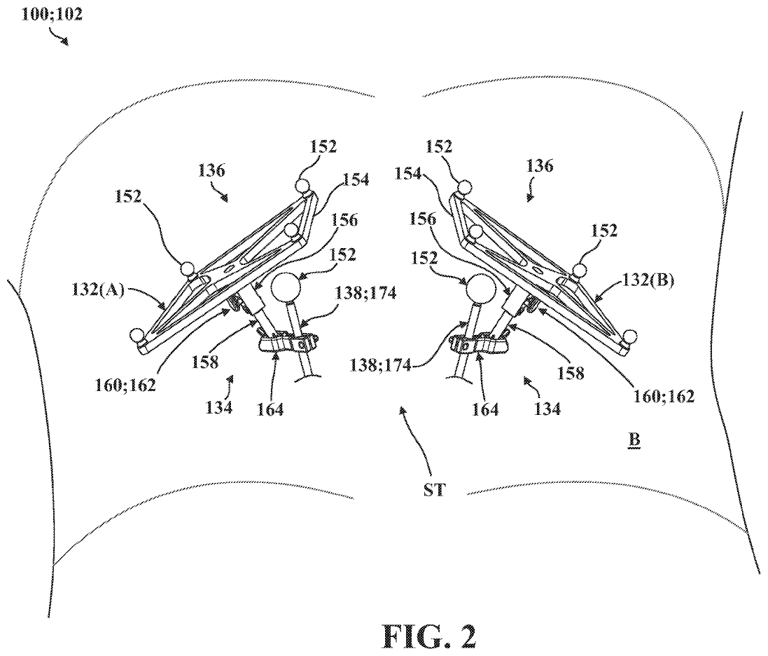

Referring now to FIGS. 1-2, as noted above, the patient trackers 132A, 132B are supported on respective mount assemblies 134 according to versions of the present disclosure which, in turn, are secured to different portions of the patient's P anatomy (e.g., on opposing lateral sides of the ilium). In the representative versions of the patient trackers 132A, 132B illustrated throughout the drawings, each of the patient trackers 132A, 132B comprises a respective array 154 to which four markers 152 are secured. As noted above, the markers 152 in this illustrative versions are realized as “passive” reflective spheres that can be removably secured to the array 154. However, those having ordinary skill in the art will appreciate that other configurations are contemplated, and the patient trackers 132A, 132B could be of various styles, types, and/or configurations, and could employ any suitable quantity, type, and/or arrangement of markers 152 without departing from the scope of the present disclosure. In some versions, the markers 152 or other portions of the trackers 132 may be similar to as is disclosed in International Patent Application No. PCT/US2021/027181, filed on Apr. 14, 2021 and entitled “Methods and Systems for Performing Image Registration In a Computer-Assisted Surgery System,” the disclosure of which is hereby incorporated by reference in its entirety. Other configurations are contemplated.

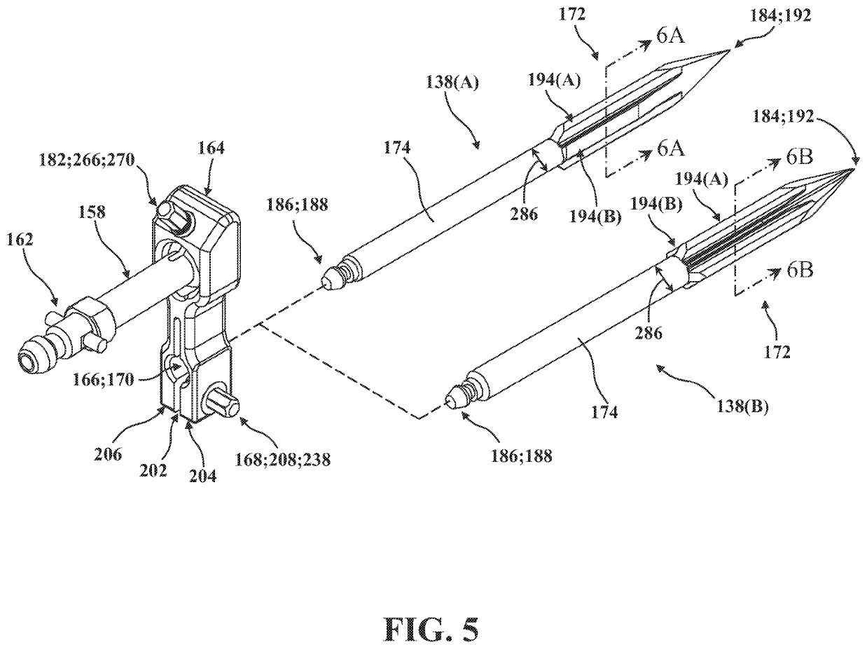

The illustrated patient trackers 132A, 132B also each comprise a dock, generally indicated at 156, that is operatively attached to the array 154 and is configured to releasably attach to a coupler 158 of the mount assembly 134 which, as is described in greater detail below, is configured to be adjustably positionable relative to the anchor 138 (and, thus, to the patient's P anatomy). As is best shown in FIG. 5, the dock 156 defines a keyed socket 160 that is shaped to receive a tracker interface 162 of the coupler 158 to effect a rigid kinematic link between the coupler 158 and the tracker 132. A portion of the dock 156 (e.g., a button, lever, and the like) may be arranged for engagement by a user (e.g., the surgeon) to effect releasing the patient tracker 132A, 132B from the coupler 158 of the mount assembly 134. While not described in detail herein, this type of dock 156 is employed in connection with “active marker” trackers described in U.S. Pat. No. 7,725,162, entitled “Surgery System,” the disclosure of which is incorporated by reference in its entirety. However, it will be appreciated that releasable attachment to the patient trackers 132A, 132B may be effected in other ways sufficient to effect a rigid kinematic link with the mount assembly 134. Moreover, it is contemplated that, in some versions, the patient tracker 132A, 132B may be fixed or otherwise secured to the coupler 158 of the mount assembly 134. Other configurations are contemplated.

It will be appreciated that utilizing markers 152 on the array 154 and on the anchor 138 can help facilitate improved operation of the navigation system 102, such as by facilitating the determination of inadvertent loosening of the tracker 132 (e.g., “bump detection”) whereby changes in the pose of the array 154 (e.g., determined based on the markers 152 attached to the array 154) can be determined relative to one or more of the anchors 138, 138B. Put differently, the navigation system 102 can monitor for changes in how one or more markers 152 coupled to the anchor 138 are arranged relative to the markers 152 coupled to the array 154, and may alert or otherwise notify users of the surgical system 100 to, among other things, check for loosening of the anchor 138 and/or of portions of the tracker 132 in the event a change in the arrangement of the markers 152 is determined. In some versions, such as is depicted in FIG. 2, separate anchors 138 could be attached to different locations on the pelvis with each anchor supporting a respective array 154 and a marker 154 coupled to the anchor 138 itself, while in other versions one anchor 138 could support an array 154 (with or without a marker 152 coupled to the anchor 138 itself), and the other anchor 138 could be utilized without an array 154 whereby a marker 152 coupled to the other anchor 138 could be used as a reference for bump detection of the array 154. However, other configurations are contemplated, and it will be appreciated that bump detection could be achieved with various styles, types, and arrangements of anchors 138, markers 152, and the like.

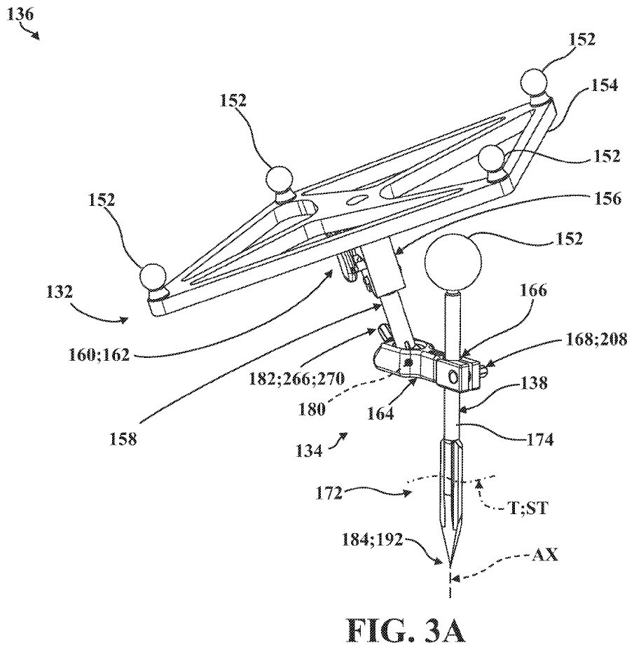

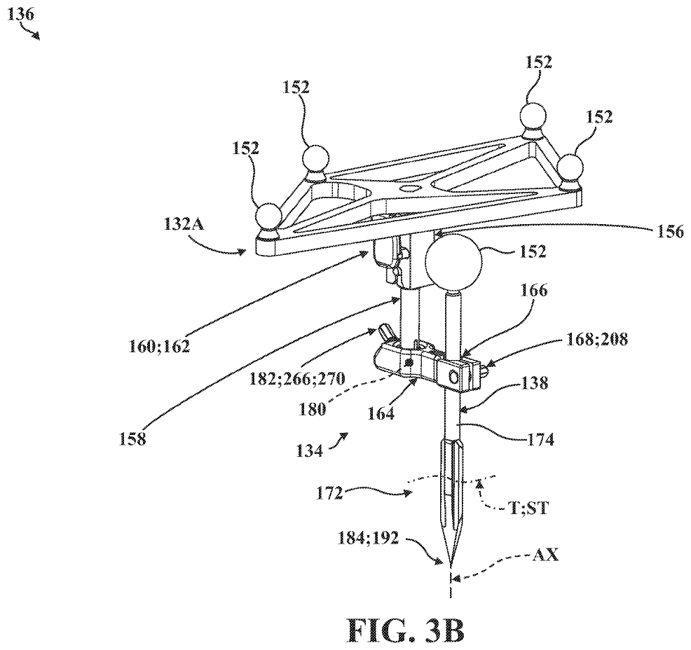

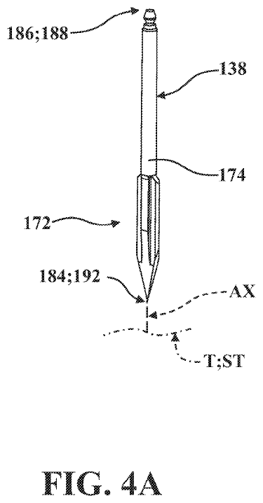

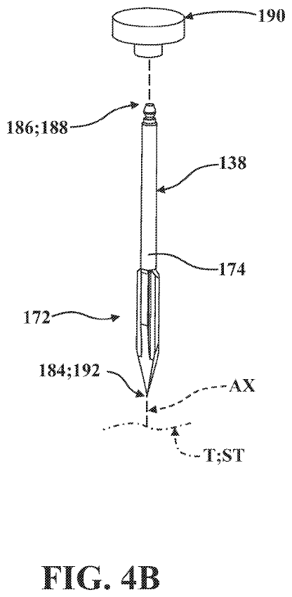

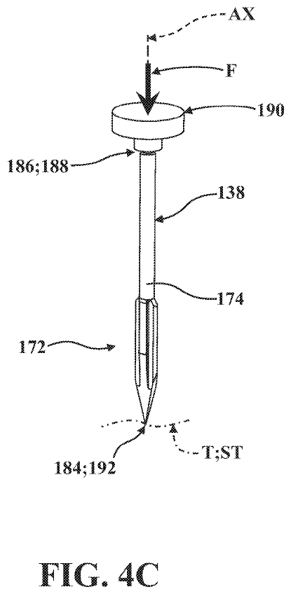

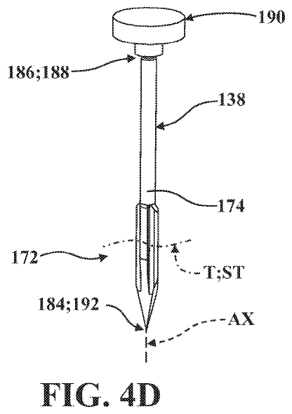

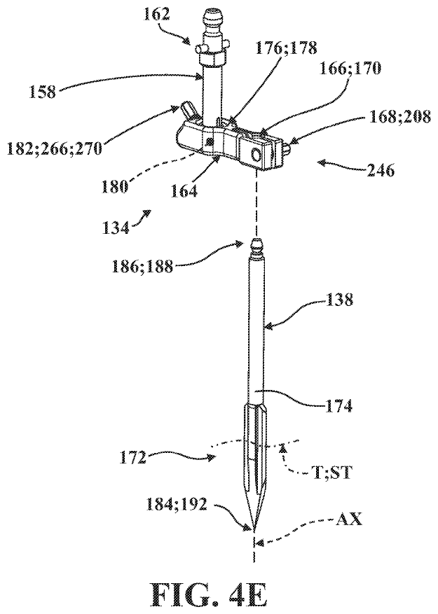

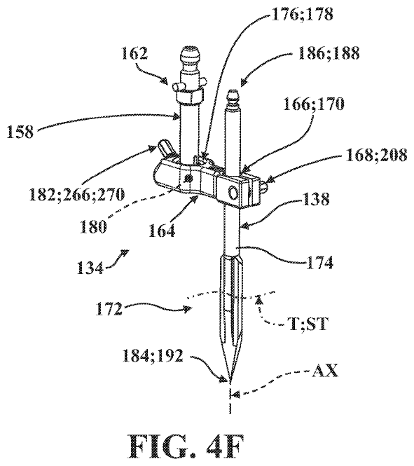

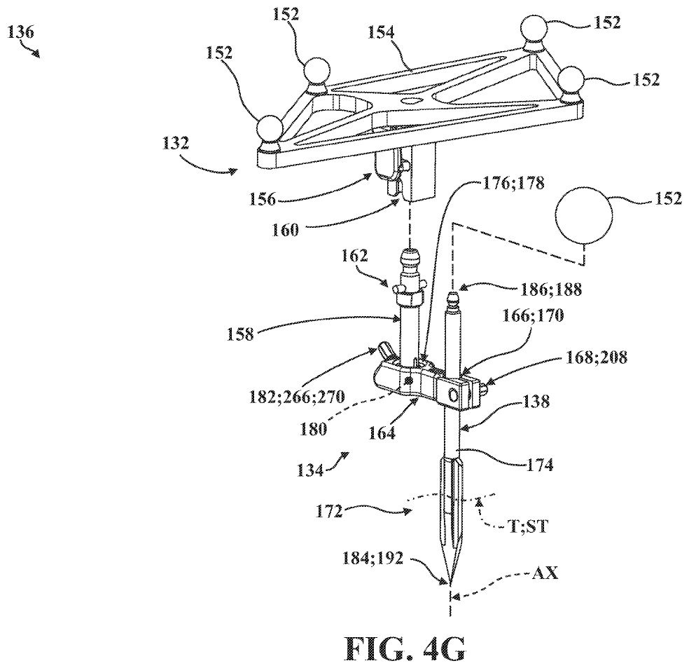

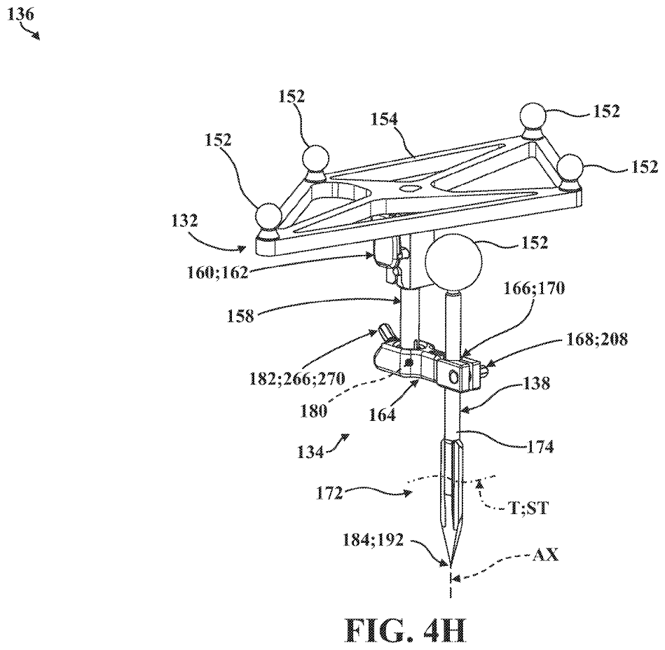

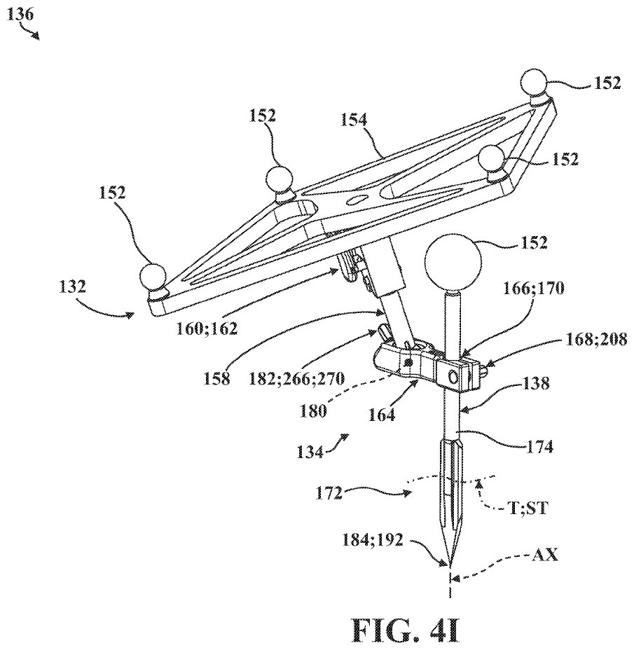

For the purposes of clarity and consistency, subsequent use herein of the term “tracker 132” refers to one of the patient trackers 132A, 132B described above, unless otherwise indicated. Referring now to FIGS. 3A-4, one of the tracker assemblies 136 is shown generally comprising the tracker 132 and the mount assembly 134 according to versions of the present disclosure. As noted above, the mount assembly 134 employs an anchor 138 to facilitate releasable attachment to tissue (e.g., bone) of the patient's P anatomy while, at the same time, affording a high level of selectively adjustable positioning of the tracker 132 relative to the tissue.

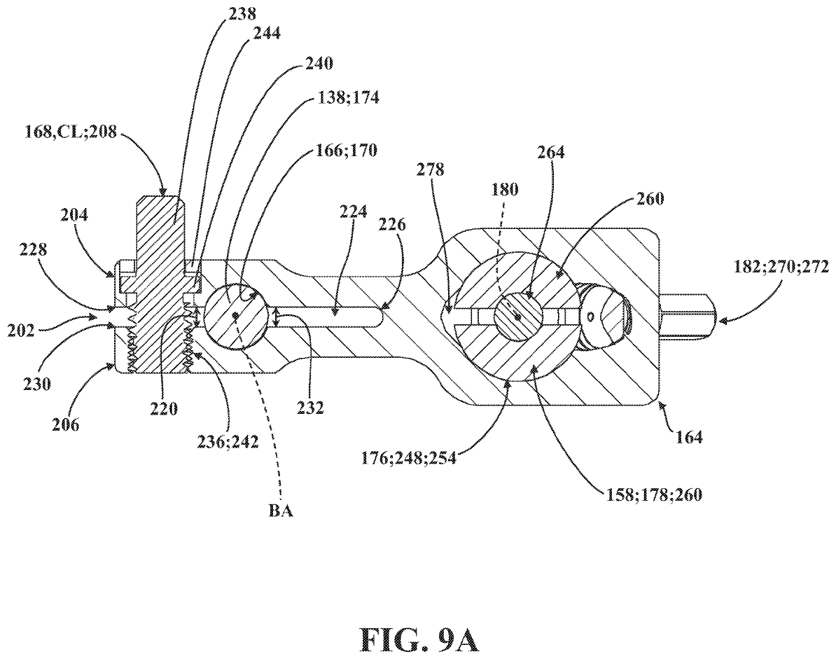

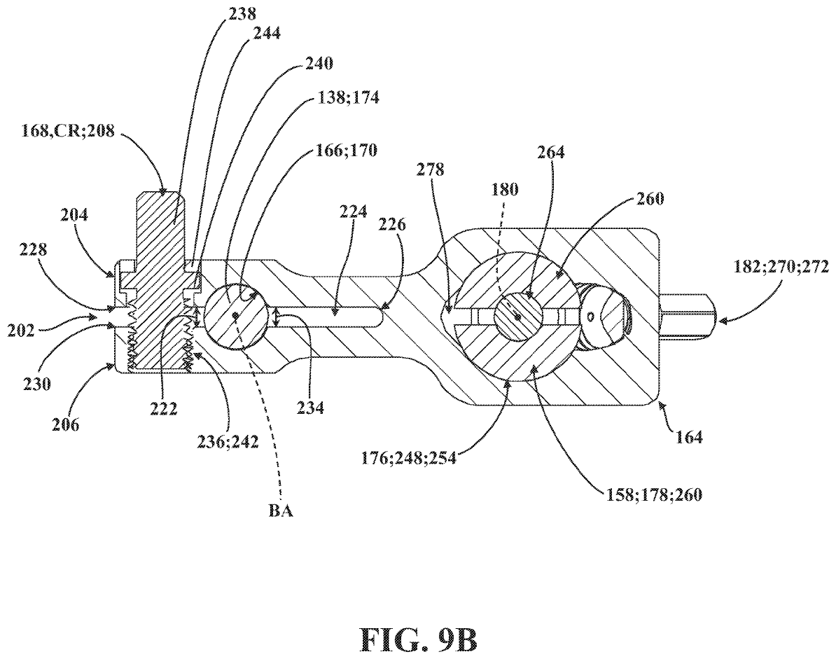

To this end, in versions of the present disclosure, the mount assembly 134 generally comprises the anchor 138, the coupler 158, a frame 164, a guide 166, and a guide lock 168. The guide 166 is operatively attached to the frame 164 defines a bore 170, and the coupler 158 is operatively attached to the frame 164 in spaced relation from the bore 170, as described in greater detail below. The coupler 158 supports the tracker 132 relative to the frame 164, such as by releasably securing the tracker 132 to the mount assembly 134 via releasable attachment of the tracker interface 162 to the dock 156. The anchor 138 has an arrow body 172 arranged for engagement with tissue T, and a shank 174 arranged for selective sliding engagement with the bore 170 of the guide 166. The guide lock 168 is operatively attached to the frame 164 and is selectively operable between: a locked configuration CL (see FIG. 9A) to restrict movement of the shank 174 along the bore 170 to effect concurrent movement of the tracker 132 with tissue T (e.g., bones of the patient's P anatomy at or adjacent to the target site ST) engaged by the anchor 138; and a released configuration CR (see FIG. 9B) to permit movement of the shank 174 along the bore 170. Each of the components of the mount assembly 134 introduced above will be described in greater detail below.

As will be appreciated from the subsequent description below, the guide lock 168 introduced above allows the user to position the frame 164 in various arrangements relative to the anchor 138 (and, thus, relative to the patient P) while in the released configuration CR, and ensures that the frame 164 (and, thus, the tracker 132) remains stationary relative to the anchor 138 while in the locked configuration CL while, at the same time, providing a compact overall profile that promotes consistent and reliable attachment of the tracker 132 to the patient's P anatomy.

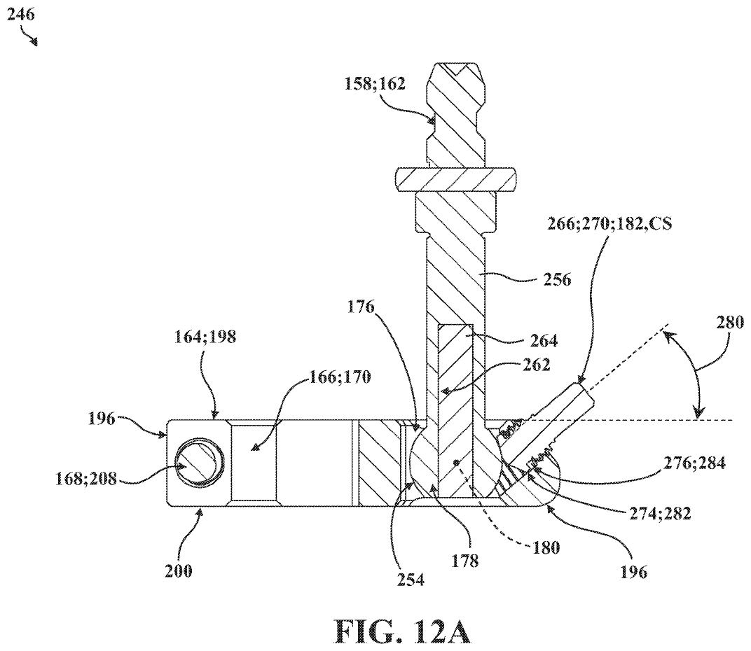

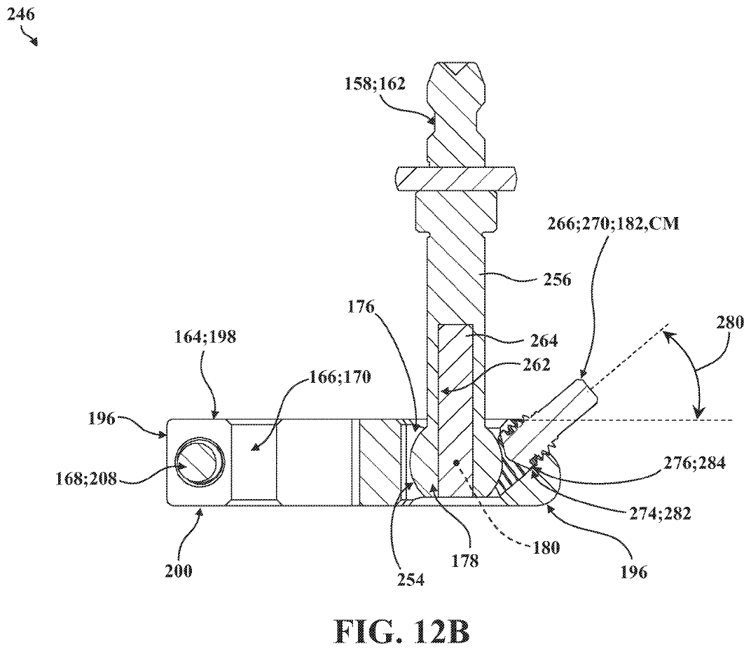

In the representative version of the mount assembly 134 illustrated herein, the coupler 158 is operatively attached to the frame 164 for releasably securing the tracker 132, as noted above, and is also configured to permit selective movement of the coupler 158 in order to allow the user to arrange the tracker 132 in different orientations relative to the frame 164. To this end, in versions of the present disclosure, the frame 164 defines a coupler seat 176 that is disposed in spaced relation from the bore 170. Here, the coupler 158 has a perch 178 that is operatively attached to and spaced from the tracker interface 162, and is arranged in the coupler seat 176 for selective movement relative to the frame 164 about a coupler point 180. A coupler lock 182 operatively attached to the frame 164 is selectively operable between: a secured configuration CS (see FIG. 12A) to restrict movement of the coupler 158 relative to the frame 164 about the coupler point 180; and a movable configuration CM (see FIG. 12B) to permit limited movement of the coupler 158 relative to the frame 164 about the coupler point 180. Each of the components of the mount assembly 134 introduced above in connection with the coupler lock 182 will be described in greater detail below.

As will be appreciated from the subsequent description below, the coupler lock 182 introduced above allows the user to position the tracker 132 in various arrangements relative to the frame 164 (and, thus, relative to the anchor 138 and the patient P) while in the movable configuration CM, and ensures that the tracker 132 remains stationary relative to the frame 164 (and, thus, relative to the anchor 138 and the patient P) while in the secured configuration CS. Here too, the pivoting of the coupler 158 effected via the perch 178 in the coupler seat 176 (when the coupler lock 182 operates in the movable configuration CM) affords a significant amount of adjustability between the tracker 132 and the frame 164 in multiple degrees of freedom (compare FIGS. 4H-4I) while, at the same time, providing a compact overall profile that promotes consistent and reliable attachment of the tracker 132 to the patient's P anatomy. As noted above, it is contemplated that mount assemblies 134 could be configured without a coupler lock 182 in certain versions, such as where the tracker 132 is rigidly coupled to the frame 164 (not shown).

In the representative versions illustrated throughout the drawings, and as is best depicted in FIG. 4A, the anchor 138 extends along an axis AX between a distal end 184 and a proximal end 186. The shank 174 is disposed along the axis AX between the distal end 184 and the proximal end 186. The proximal end 186 is arranged for receiving impact force F, such as from a hammer or other tool, instrument, and the like. The distal end 184 is arranged for engaging tissue T (e.g., bones of the patient's P anatomy at or adjacent to the target site ST) as described in greater detail below. In the illustrated version, the anchor 138 includes a head 188 coupled to (or otherwise formed as a part of) the shank 174 and arranged at the proximal end 186 for receiving impact force F to advance the arrow body 172 into engagement with tissue T. In some versions, impact force F may be applied directly to the proximal end 186 (e.g., to the head 188). In some versions, an impaction receiver 190 (see FIGS. 4C-4D; not shown in detail) may be removably coupled to the head 188 adjacent to the proximal end 186 of the anchor 138 to receive impact force F. In some versions, the head 188 of the anchor 138 may be configured to releasably engage markers 152 (see FIGS. 4G-4H). Other configurations are contemplated.

The proximal end 186 of the anchor 138 is shaped to enter into and pass through the bore 170 of the guide 166 to bring the shank 174 into sliding engagement with the bore 170 when the guide lock 168 operates in the released configuration CR. The shank 174 has a substantially cylindrical profile configured for sliding engagement with the bore 170 of the guide 166 such that the transfer of force, torque, and the like applied to the shank 174 is at least partially prevented from being transferred to the guide 166 (and, thus, the frame 164) when the guide lock 168 operates in the released configuration CR. Thus, with this configuration, the user can move the frame 164 along the shank 174 translationally and/or rotationally after the anchor 138 is impacted into tissue T. Furthermore, with this configuration, it is also contemplated that the user could position the frame 164 along the shank 174 to impact the anchor 138, either with the guide lock 168 in the released configuration CR (e.g., such that the anchor 138 advances into tissue T while the shank 174 is slidably supported by the bore 170 of the guide 166), or with the guide lock 167 in the locked configuration CL (e.g., such that the anchor 138 advances into tissue T concurrently with the guide 166, the frame 164, and the like.

The arrow body 172 is coupled to the shank 174 and has a tip 192 and a pair of wing braces 194. The tip 192 tapers towards the distal end 184 for advancing into engagement with tissue T. As is described in greater detail below, the wing braces 194 extend away from each other and extend transverse to the axis AX to inhibit rotation of the anchor 138 about the axis AX relative to engaged tissue T.

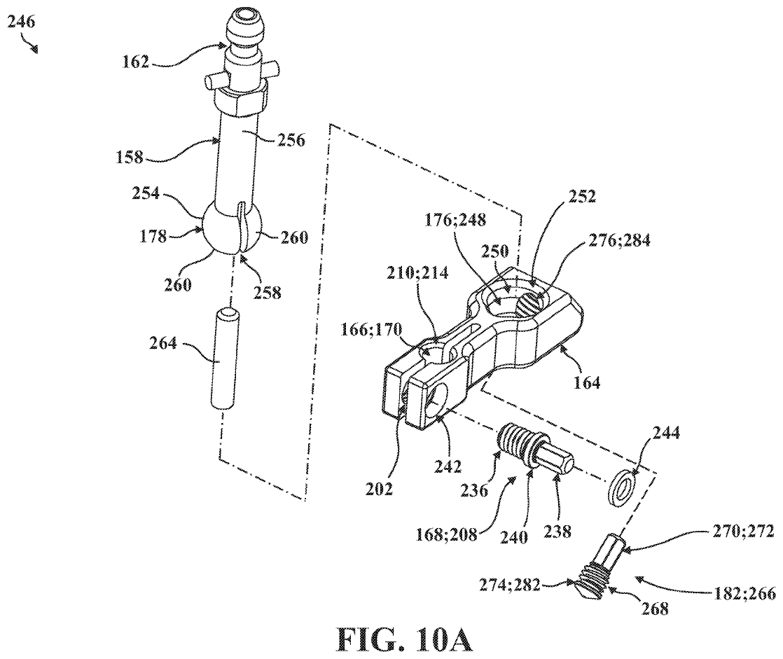

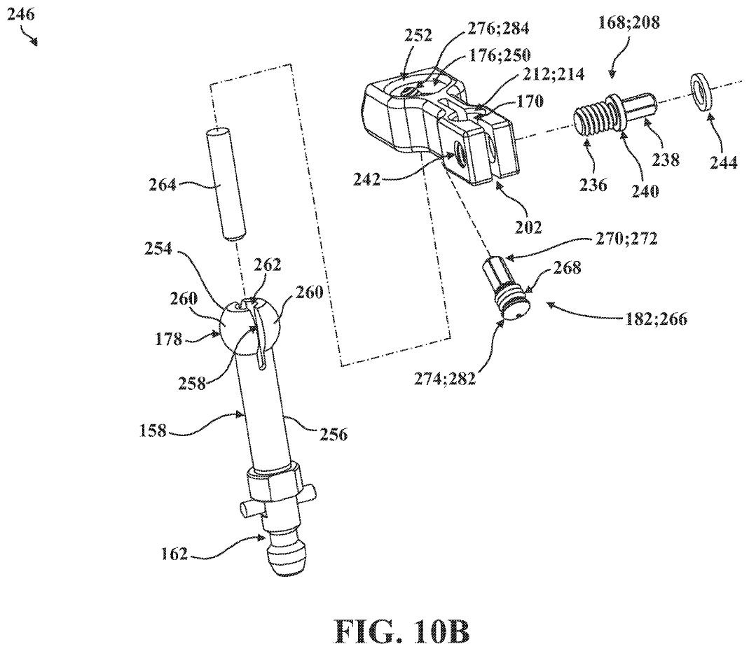

Referring now to FIG. 5, as noted above, the frame 164 of the mount assembly 134 defines the coupler scat 176, and the guide 166 is operatively attached to the frame 164 and defines the bore 170. In the representative version illustrated herein, the guide 166 is formed as a part of the frame 164. Put differently, the frame 164 defines the bore 170 in the illustrated versions. However, it will be appreciated that the guide 166 could be formed as a separate component which is operatively attached to the frame 164. The frame 164 has an outer frame surface 196, an upper frame surface 198, and a lower frame surface 200. A frame slot, generally indicated at 202, is formed extending through the upper and lower frame surfaces 198, 200 and extends into the bore 170 to define first and second flexure portions 204, 206 which facilitate operation of the guide lock 168 between the locked and released configurations CL, CR in the illustrated versions. To this end, the representative version of the guide lock 168 includes a guide retainer 208 operatively attached to the frame 164 to urge the first and second flexure portions 204, 206 towards each other in response to changing operation from the released configuration CR to the locked configuration CL (compare FIGS. 9A-9B; movement of flexure portions 204, 206 not shown in detail). Here, the first and second flexure portions 204, 206 of the frame 164 are arranged or otherwise configured to resiliently move away from each other in response to changing operation from the locked configuration CL to the released configuration CR. As is best shown in FIGS. 10A-10B, the bore 170 has an inlet 210, an outlet 212, and tapered regions 214 adjacent to the inlet 2018 and the outlet 212 which merge with the upper and lower frame surfaces 198, 200. The tapered regions 214 have a generally frustoconical profile shaped and arranged to facilitate smooth engagement between the shank 174 and the bore 170.

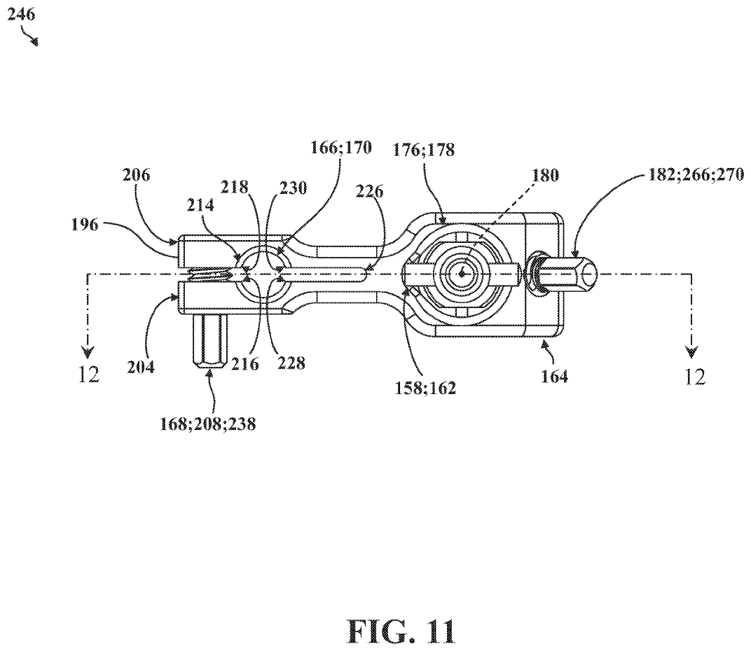

The frame slot 202 is formed through the outer frame surface 196 extending into the bore 170 to define first and second bore edges 216, 218 (see FIG. 11), which are arranged substantially parallel to the bore axis BA. At least a portion of the first and second bore edges 216, 218 are spaced from each other at a first bore edge distance 220 when the guide lock 168 operates in the locked configuration CL (see FIG. 9A), and at a second bore edge distance 222 larger than the first bore edge distance 220 when the guide lock 168 operates in the released configuration CR (see FIG. 9B, compare to FIG. 9A; distances 220, 222 not shown in detail). Here, the first and second flexure portions 204, 206 act to “pinch” the shank 174 within the bore 170 to inhibit movement of the anchor 138 when the guide lock 168 operates in the locked configuration CL.

In some versions, a relief slot 224 is formed in the frame 164 at a location spaced from the frame slot 202. The relief slot 224 is similarly formed through the upper and lower frame surfaces 198, 200, and extends from a relief slot end 226 (see FIGS. 9A-9B) into the bore 170 to define first and second relief slot edges 228, 230 (see FIG. 11) which likewise have profiles complimentary to (and generally extend parallel relative to) the bore 170. At least a portion of the first and second relief slot edges 228, 230 are spaced from each other at a first relief edge distance 232 when the guide lock 168 operates in the locked configuration CL (see FIG. 9A), and at a second relief edge distance 234 larger than the first relief edge distance 232 when the guide lock 168 operates in the released configuration CR (see FIG. 9B, compare to FIG. 9A; distances 232, 234 not shown in detail). The relief slot 224 is spaced radially about the bore 170 from the frame slot 202 so as to further delineate the first and second flexure portions 204, 206 from each other while, at the same time, affording the frame 164 with a compact profile. It will be appreciated that the relief slot 224 helps distribute force about the bore 170 between the first and second flexure portions 204, 206 in order to minimize the amount of force which needs to be applied to the guide retainer 208 by the user in order to move the guide lock 168 between the locked and released configurations CL, CR.

As noted above, the guide retainer 208 is operatively attached to the frame 164 to urge the first and second flexure portions 204, 206 towards each other in response to changing operation from the released configuration CR to the locked configuration CL (compare FIGS. 9A-9B). To this end, in the illustrated versions, and as is best shown in FIG. 10A, the guide retainer 208 includes a retention portion 236, a guide interface 238 arranged for engagement by the user to operate the guide lock 168 between the locked and released configurations CL, CR, and a guide retainer body 240 extending between the retention portion 236 and the guide interface 238. The frame 164 defines a guide retainer aperture 242 formed extending through the first and second flexure portions 204, 206 that is arranged to receive the guide retainer body 240. Here, the guide retainer 208 is realized as a threaded fastener with an elongated hex-shaped head defining the guide interface 238, a shaft defining the guide retainer body 240, and threads defining the retention portion 236. The guide interface 238 is shaped to receive torque from a tool (e.g., a socket, wrench, or similar fastener driver; not shown). At least a portion of the guide retainer aperture 242 (e.g., internal threads formed in part of one of the first and second flexure portions 204, 206) is disposed in threaded engagement with at least a portion of the retention portion 236 (e.g., external threads) such that rotational torque applied to the guide interface 238 in one direction urges the first and second flexure portions 204, 206 towards each other to operate the guide lock 168 in the locked configuration CL, and such that rotational torque applied to the guide interface 238 in an opposite direction permits movement of the first and second flexure portions 204, 206 away from each other to operate the guide lock 168 in the released configuration CR. It will be appreciated that the guide retainer 208 could be realized or otherwise configured in other ways (e.g., other than as a threaded fastener) to change operation of the guide lock 168 by facilitating relative movement between the first and second flexure portions 204, 206. In the illustrated version, the guide retainer body 240 is constrained relative to the frame 164 via an interface ring 244, which may be pressed into the guide retainer aperture 242 after the guide retainer 208 is inserted.

Referring now to FIGS. 5 and 10A-10B, the coupler 158, the frame 164, the guide 166, and the guide lock 168 cooperate to define a frame subassembly 246 which, together with the anchor 138, defines the mount assembly 134 according to versions of the present disclosure. The coupler 158 is disposed in sliding contact with the coupler seat 176 of the frame 164. Here, and as is best shown in FIG. 10A, the coupler seat 176 has a curved coupler region 248, transition coupler regions 250 extending from the curved coupler region 248, and tapered coupler regions 252 extending from the transition coupler regions 250 to the upper and lower frame surfaces 198, 200. Here, the curved coupler region 248 has a generally spherical profile to facilitate pivoting movement of the perch 178 of the coupler 158 about the coupler point 180 (compare FIGS. 4H-4I). The transition coupler regions 250 have a generally cylindrical profile that is sized and arranged to facilitate retention of the perch 178 within the coupler seat 176. The tapered coupler regions 252 have a generally frustoconical profile that are shaped and arranged to facilitate pivoting of the coupler 158 about the coupler point 180.

The perch 178 of the coupler 158 has a perch pivot surface 254 that is disposed in sliding contact with the curved coupler region 248 of the coupler seat 176 to facilitate selective pivoting movement of the coupler 158 about the coupler point 180 when the coupler lock 182 operates in the movable configuration CM. Here too, the perch pivot surface 254 has a generally spherical profile. In the illustrated version, the coupler 158 has a brace 256 extending between the tracker interface 162 and the perch 178. As is best shown in FIG. 10B, a perch slot 258 is formed extending through the perch pivot surface 254 to define coupler flexure regions 260, and a keeper bore 262 is formed through each of the coupler flexure regions 260 and into the brace 256 to receive a keeper shaft 264. Here, the keeper shaft 264 is inserted into the keeper bore 262 after the perch 178 has been arranged into the coupler scat 176 in order to retain the coupler 158 to the frame 164 even while the coupler lock 182 operates in the movable configuration CM while permitting limited rotation of the perch 178 in three degrees of freedom about the coupler point 180. Put differently, without the keeper shaft 264, the coupler flexure regions 260 are able to resiliently move relative to each other to facilitate installation into the coupler seat 176.

Referring now to FIGS. 10A-12B, the illustrated coupler lock 182 includes a coupler retainer 266 that is supported by the frame 164 and is arranged to abut the perch 178 of the coupler 158 when the coupler lock 182 operates in the secured configuration CS (see FIG. 12A). To this end, and as is best shown in FIG. 10A, the coupler retainer 266 includes a coupler retention portion 268, a coupler interface 270 arranged for engagement by the user to operate the coupler lock 182 between the secured and movable configurations CS, CM, and a coupler retainer body 272 extending between the coupler retention portion 268 and the coupler interface 270 to a coupler end portion 274 arranged to abut the perch 178. The frame 164 defines a coupler retainer aperture 276 formed extending into communication with the coupler seat 176 (e.g., into the outer frame surface 196) that is arranged to receive the coupler retainer body 272. Here too, the coupler retainer 266 is realized as a threaded fastener with an elongated hex-shaped head defining the coupler interface 270, and has a shaft defining the coupler retainer body 272 extending to the coupler end portion 274 with threads defining the coupler retention portion 268. The coupler interface 270 is similarly shaped to receive torque from a tool (e.g., a socket, wrench, or similar fastener driver; not shown). At least a portion of the coupler retainer aperture 276 (e.g., internal threads) is disposed in threaded engagement with at least a portion of the coupler retention portion 268 (e.g., external threads) such that rotational torque applied to the coupler interface 270 in one direction urges the coupler end portion 274 into abutment with the perch 178 of the coupler 158 to operate the coupler lock 182 in the secured configuration CS (see FIG. 12A), and such that rotational torque applied to the coupler interface 270 in an opposite direction urges the coupler end portion 274 out of abutment with the perch 178 to operate the coupler lock 182 in the movable configuration CM (see FIG. 12B). The coupler seat 176 has an ovate profile defining a reduced coupler region 278 (see FIGS. 9A-9B), which allows the perch 178 to flex within the coupler seat 176 as the coupler lock 182 is moved to the secured configuration CS.

In the illustrated version, the coupler retainer 266 of the coupler lock 182 (as well as the coupler retainer aperture 276) is arranged at an oblique coupler angle 280 relative to the upper frame surface 198 (see FIG. 12A). This configuration helps promote access to the coupler retainer 266 from above during use. The coupler interface 270 is configured with a profile that is narrow enough to pass into and through the coupler retainer aperture 276 so as to be installed into the frame 164 from “beneath” (see FIG. 10B) before the perch 178 is inserted into the coupler seat 176. The coupler retainer 266 is provided with a flange 282 that limits how far the coupler retainer 266 can travel along the coupler retainer aperture 276. Here, the coupler retainer aperture 276 is provided with an undercut 284 sized to accommodate the flange 282 but also limit travel of the coupler retainer 266 once installed. Once the coupler retainer 266 is inserted from “beneath” and the perch 178 is inserted, the coupler retainer 266 can be adjusted via rotation, but cannot be removed out of the frame 164 without also removing the perch 178 due to the flange 282. This configuration affords opportunities for improved handling and retention of the coupler retainer 266 by preventing inadvertent removal from the frame 164 during adjustment, handling, and the like.

It will be appreciated that the coupler retainer 266 could be realized or otherwise configured in other ways (e.g., other than as a threaded fastener) to change operation of the coupler lock 182 by facilitating abutment with the perch 178 or otherwise inhibiting movement of the perch 178 relative to the frame 164.

Referring now to FIGS. 5-7B, as noted above, the arrow body 172 of the anchor 138 employs the tip 192 and the pair of wing braces 194 for advancing into engagement with tissue T and for inhibiting rotation of the anchor 138 about the axis AX relative to engaged tissue T. Here, the shank 174 defines a shank diameter 286 (see FIG. 5) adjacent to the arrow body 172, and the wing braces 194 extend away from the axis AX to respective wing brace ends 288 spaced from each other at a wing brace distance 290 that is larger than the shank diameter 286. As will be appreciated from the subsequent description below, various configurations of the arrow body 172 are contemplated by the present disclosure, including versions which employ a single pair of wing braces 194.

In the representative versions of the anchor 138 illustrated herein, the arrow body 172 includes a first pair of wing braces 194A which extend to respective first wing brace ends 288A spaced from each other at a first wing brace distance 290A, and a second pair of wing braces 194B which extend to respective second wing brace ends 288B spaced from each other at a second wing brace distance 290B. The second pair of wing braces 194B are interposed radially about the axis AX between the first pair of wing braces 194A and extend away from each other transverse to the axis AX to inhibit rotation of the anchor 138 about the axis AX relative to engaged tissue T. In the illustrated versions, the first pair of wing braces 194A extend generally laterally away from each other (e.g., away from the axis AX), and the second pair of wing braces 194B similarly extend generally laterally away from each other (e.g., away from the axis AX) and are “clocked” perpendicularly about the axis AX relative to the first pair of wing braces 194A (e.g., radially interposed at 90-degrees).

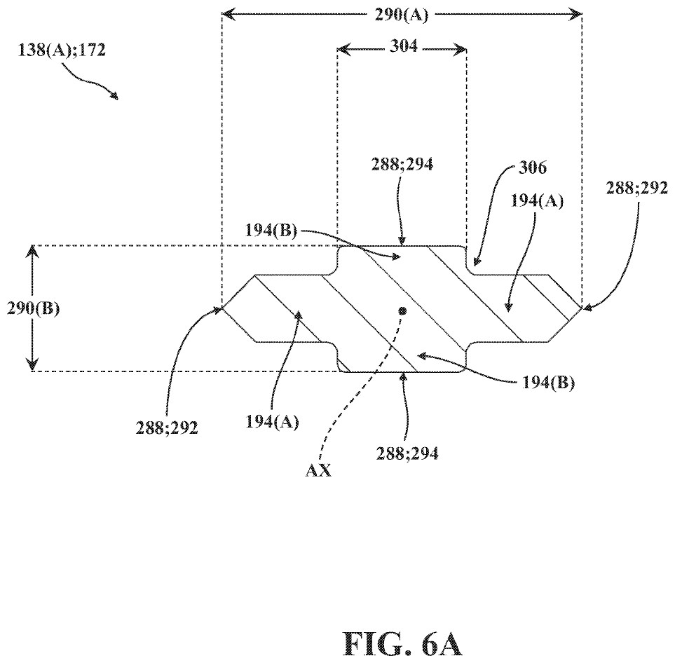

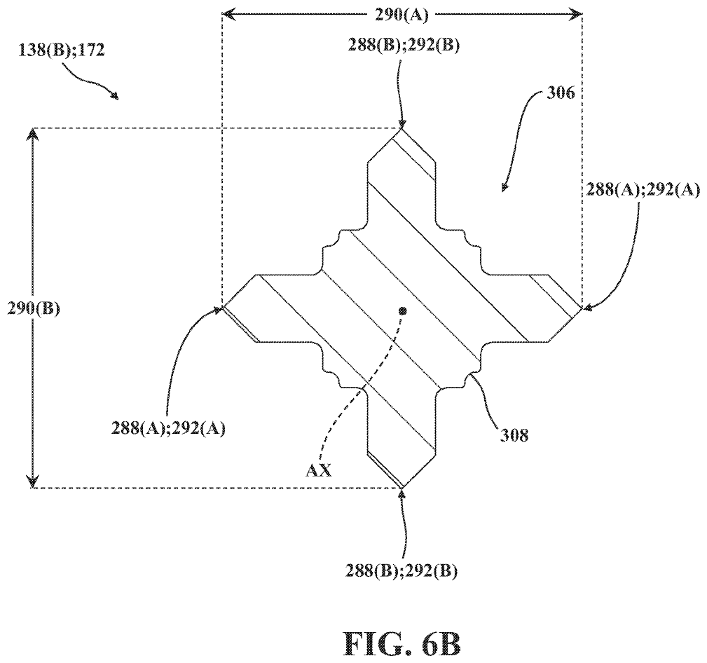

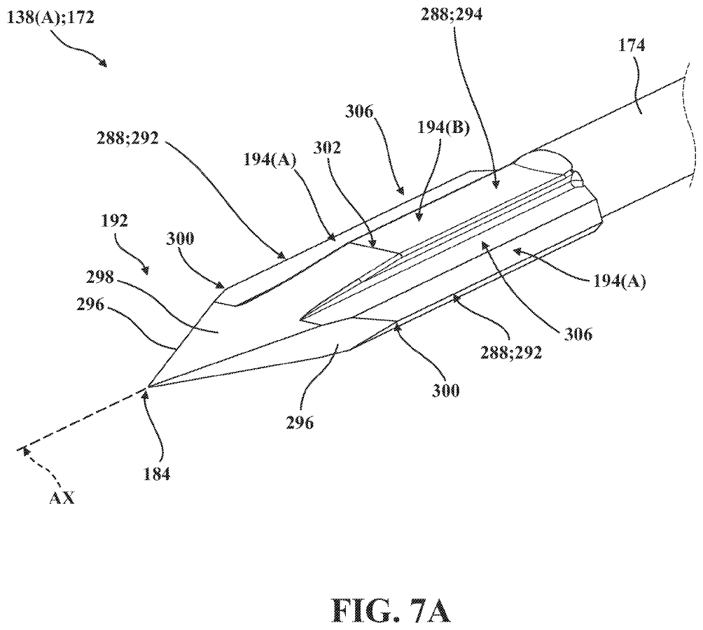

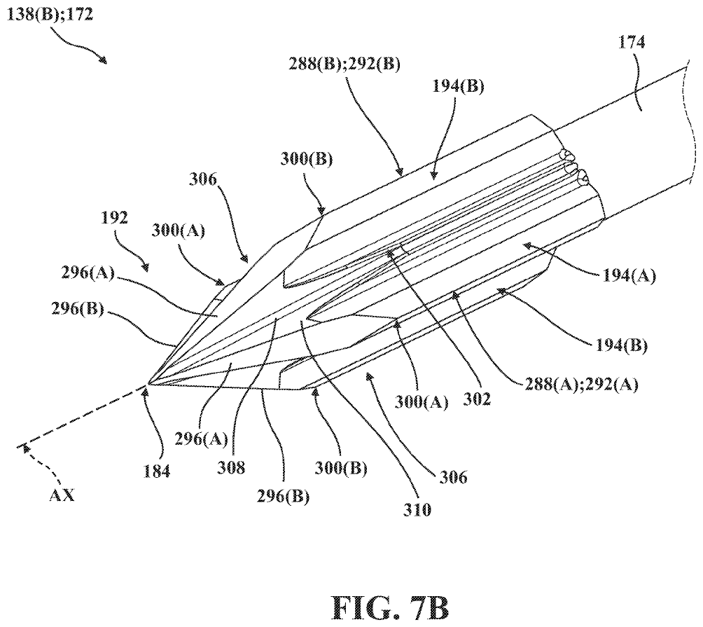

FIG. 5 depicts a first version of the anchor 138A (see also FIGS. 6A and 7A) and a second version of the anchor 138B (see also FIGS. 6B and 7B). In the first version of the anchor 138A depicted in FIGS. 6A and 7A, the first pair of wing brace ends 288A have tapered profiles and are further defined as a pair of wing brace tips 292, the second pair of wing brace ends 288B have flat profiles and are further defined as a pair of wing brace edges 294, and the first wing brace distance 290A is larger than the second wing brace distance 290B. However, in the second version of the anchor 138B depicted in FIGS. 6B and 7B, the first and second pairs of wing brace ends 288A, 288B each have tapered profiles and are further defined as first and second pairs of wing brace tips 292A, 292B, and the first wing brace distance 290A is substantially equivalent to the second wing brace distance 290B. However, as noted above, other configurations of the arrow body 172 are contemplated by the present disclosure, including with various types, styles, and arrangements of wing braces 194, which may be configured as wing brace tips 292, as wing brace edges 294, or with other profiles.

Referring now to FIGS. 6A and 7A, the illustrated version of the anchor 138A includes a pair of tip transition surfaces 296 and a pair of edge transition surfaces 298. The tip transition surfaces 296 extend between the tip 192 and the pair of wing brace tips 292, and the pair of edge transition surfaces 298 extend between the tip 192 and the pair of wing brace edges 294. Here too in this version, the arrow body 172 includes a pair of tip apexes 300 and a pair of edge apexes 302 (see FIG. 7A; not shown in detail). The pair of tip apexes 300 are defined between the pair of tip transition surfaces 296 and the pair of wing brace tips 292, and the pair of edge apexes 302 are defined between the pair of edge transition surfaces 298 and the pair of wing brace edges 294. In this version, the pair of edge apexes 302 are spaced longitudinally along the axis AX between the pair of tip apexes 300 and the proximal end 186. Put differently, the tip apexes 300 are arranged closer than the edge apexes 302 to the distal end 184. Here in this version, the wing brace edges 294 each have a substantially flat profile, and define a lateral edge width 304 (see FIG. 6A) taken adjacent to the edge apexes 302. The lateral edge width 304 is smaller than the first wing brace distance 290A (as well as the shank diameter 286), and may be substantially equivalent to the second wing brace distance 290B. In the illustrated version, the configuration of the lateral edge width 304 and arrangement of the edge apexes 302 relative to the distal end 184 gives the edge transition surfaces 298 a shallow, flat profile that is asymmetric relative to the tip transition surfaces 296. It will be appreciated that this configuration affords significant surface area contact with tissue T and can help facilitate reliable, accurate engagement of tissue T transitioning from the tip 192 to the wing braces 194.

Referring now to FIGS. 6B and 7B, the illustrated version of the anchor 138B includes first and second pairs of tip transition surfaces 296A, 296B. The first pair of tip transition surfaces 296A extend between the tip 192 and the first pair of wing brace tips 292A, and the second pair of tip transition surfaces 296B extend between the tip 192 and the second pair of wing brace tips 292B. In this version, the arrow body 172 includes first and second pairs of tip apexes 300A, 300B (see FIG. 7B; not shown in detail). The first pair of tip apexes 300A are defined between the first pair of tip transition surfaces 296A and the first pair of wing brace tips 292A, and the second pair of tip apexes 300B are defined between the second pair of tip transition surfaces 296B and the second pair of wing brace tips 292B. The first and second pairs of tip apexes 300A, 300B are each spaced longitudinally along the axis AX between the shank 174 and the distal end 184. Put differently, the first pair of tip apexes 300A are not spaced closer than the second pair of tip apexes 300B to the distal end 184. In the illustrated version, the configuration of the first and second wing brace distances 290A, 290B and arrangement of the first and second pair of tip apexes 300A, 300B relative to the distal end 184 gives the first and second tip transition surfaces 296 a symmetrically-spaced profile that likewise affords significant surface area contact with tissue T and can help facilitate reliable, accurate engagement of tissue T transitioning from the tip 192 to the wing braces 194.

In the versions of the anchor 138 depicted in FIGS. 6A-7B, nocks 306 are defined interposed radially between the first and second pairs of wing braces 194A, 194B. Here, the nocks have generally L-shaped profiles defined by the first and second pairs of wing braces 194A, 194B, and are arranged to accommodate tissue T after the anchor 138 has been impacted. The nocks 306 are formed extending towards the shank 174 from a location arranged longitudinally between the distal end 184 and the pair of tip apexes 300. In the version of the anchor 138B depicted in FIGS. 6B and 7B, flutes 308 are arranged along the nocks 306 and are formed extending towards the shank 174 from the distal end 184. In this version, flute transitions 310 are disposed tapering outwardly relative to the axis AX from the distal end 184 to a longitudinal location adjacent to and distal from the first and second pairs of tip apexes 300A, 300B before tapering inwardly relative to the axis AX therefrom to a more proximal longitudinal location disposed between the first and second pairs of tip apexes 300A, 300B and the shank 174 (see FIG. 7B; not shown in detail).

Those having ordinary skill in the art will appreciate that aspects of the versions described and illustrated herein can be interchanged or otherwise combined.

It will be further appreciated that the terms “include,” “includes,” and “including” have the same meaning as the terms “comprise,” “comprises,” and “comprising.” Moreover, it will be appreciated that terms such as “first,” “second,” “third,” and the like are used herein to differentiate certain structural features and components for the non-limiting, illustrative purposes of clarity and consistency.

Several configurations have been discussed in the foregoing description. However, the configurations discussed herein are not intended to be exhaustive or limit the invention to any particular form. The terminology which has been used is intended to be in the nature of words of description rather than of limitation. Many modifications and variations are possible in light of the above teachings and the invention may be practiced otherwise than as specifically described.

The present disclosure also comprises the following clauses, with specific features laid out in dependent clauses, that may specifically be implemented as described in greater detail with reference to the configurations and drawings above.

### CLAUSES

I. A mount assembly for use with a navigable tracker, the mount assembly comprising:
a frame;
a coupler operatively attached to the frame for releasably securing the tracker;
an anchor extending along an axis between a distal end for engaging tissue and a proximal end arranged to receive impaction force, the anchor including:
   a shank disposed along the axis between the distal end and the proximal end, and
   an arrow body coupled to the shank and having a tip tapering towards the distal end for advancing into engagement with tissue with a pair of wing braces extending away from the axis to inhibit rotation of the anchor about the axis relative to engaged tissue;
a guide operatively attached to the frame and defining a bore shaped to receive the shank of the anchor; and
a guide lock operable between:
   a released configuration to permit movement of the shank along the bore, and
   a locked configuration to restrict movement of the shank along the bore to effect concurrent movement of the tracker with the tissue engaged by the anchor.
II. The mount assembly as set forth in clause I, wherein the shank defines a shank diameter; and
wherein the pair of wing braces extend away from the axis to respective wing brace ends spaced from each other at a wing brace distance larger than the shank diameter.
III. The mount assembly as set forth in any of clauses I-II, wherein the pair of wing braces are further defined as a first pair of wing braces; and
wherein the arrow body further includes a second pair of wing braces interposed radially between the first pair of wing braces and extending away from each other transverse to the axis to inhibit rotation of the anchor about the axis relative to engaged tissue.
IV. The mount assembly as set forth in clause III, wherein the first pair of wing braces extend transverse to the axis to respective first wing brace ends spaced from each other at a first wing brace distance; and
wherein the second pair of wing braces extend transverse to the axis to respective second wing brace ends spaced from each other at a second wing brace distance.
V. The mount assembly as set forth in clause IV, wherein the first wing brace distance is larger than the second wing brace distance.
VI. The mount assembly as set forth in any of clauses IV-V, wherein the first pair of wing brace ends are further defined as a pair of wing brace tips; and
wherein the second pair of wing brace ends are further defined as a pair of wing brace edges.
VII. The mount assembly as set forth in clause VI, wherein the arrow body further includes a pair of tip transition surfaces extending between the tip and the pair of wing brace tips, and a pair of edge transition surfaces extending between the tip and the pair of wing brace edges.
VIII. The mount assembly as set forth in clause VII, wherein the arrow body further includes a pair of tip apexes defined between the pair of tip transition surfaces and the pair of wing brace tips, and a pair of edge apexes defined between the pair of edge transition surfaces and the pair of wing brace edges; and
wherein the pair of edge apexes are spaced longitudinally along the axis between the pair of tip apexes and the proximal end.
IX. The mount assembly as set forth in any of clauses IV-VIII, wherein the first wing brace distance is substantially equivalent to the second wing brace distance.
X. The mount assembly as set forth in any of clauses IV-IX, wherein the first pair of wing brace ends are further defined as a first pair of wing brace tips; and
wherein the second pair of wing brace ends are further defined as a second pair of wing brace tips.
XI. The mount assembly as set forth in clause X, wherein the arrow body further includes a first pair of tip transition surfaces extending between the tip and the first pair of wing brace tips, and a second pair of tip transition surfaces extending between the tip and the second pair of wing brace tips.
XII. The mount assembly as set forth in clause XI, wherein the arrow body further includes a first pair of tip apexes defined between the first pair of transition surfaces and the first pair of wing brace tips, and a second pair of tip apexes defined between the second pair of transition surfaces and the second pair of wing brace tips; and
wherein the first pair of tip apexes and the second pair of tip apexes are each spaced longitudinally along the axis between the shank and the distal end.
XIII. The mount assembly as set forth in any of clauses I-XII, wherein the frame has an outer frame surface arranged adjacent to the bore, with a frame slot extending through the outer frame surface and into the bore to define first and second bore edges.
XIV. The mount assembly as set forth in clause XIII, wherein the first and second bore edges are spaced from each other at a first bore edge distance when the guide lock operates in the locked configuration, and at a second bore edge distance when the guide lock operates in the released configuration.
XV. The mount assembly as set forth in any of clauses XIII-XIV, wherein the frame slot defines first and second flexure portions; and
wherein the guide lock includes a guide retainer operatively attached to the frame to urge the first and second flexure portions towards each other in response to changing operation from the released configuration to the locked configuration.
XVI. The mount assembly as set forth in clause XV, wherein the first and second flexure portions of the frame are arranged to resiliently move away from each other in response to changing operation from the locked configuration to the released configuration.
XVII. The mount assembly as set forth in any of clauses XV-XVI, wherein the guide retainer includes a retention portion, a guide interface arranged for engagement by a user to operate the guide lock between the locked configuration and the released configuration, and a guide retainer body extending between the retention portion and the guide interface.
XVIII. The mount assembly as set forth in clause XVII, wherein the frame further defines a guide retainer aperture formed extending through the first and second flexure portions and arranged to receive the guide retainer body.
XIX. The mount assembly as set forth in clause XVIII, wherein at least a portion of the guide retainer aperture is disposed in threaded engagement with at least a portion of the retention portion such that rotational torque applied to the guide interface in one direction urges the first and second flexure portions towards each other to operate the guide lock in the locked configuration, and such that rotational torque applied to the guide interface in an opposite direction permits movement of the first and second flexure portion away from each other to operate the guide lock in the released configuration.
XX. The mount assembly as set forth in any of clauses I-XIX, wherein the proximal end of the shank is shaped to enter into and pass through the bore of the frame to bring the shank into sliding engagement with the bore when the guide lock operates in the released configuration.
XXI. The mount assembly as set forth in clause XX, wherein the anchor includes a head coupled to the shank and arranged at the proximal end for receiving impact force to advance the arrow body into engagement with tissue.
XXII. The mount assembly as set forth in clause XXI, wherein the shank of the anchor has a generally cylindrical profile disposed in engagement with the bore of the guide such that rotational torque applied to the anchor effects rotation of the shank relative to the frame when the guide lock operates in the released configuration.
XXIII. The mount assembly as set forth in any of clauses I-XXII, wherein the coupler includes a perch arranged for selective movement relative to the frame, and a tracker interface spaced from the perch for releasably securing the tracker.
XXIV. The mount assembly as set forth in clause XXIII, wherein the frame further defines a coupler seat supporting the perch for selective movement relative to the frame about a coupler point.
XXV. The mount assembly as set forth in clause XXIV, further comprising a coupler lock operatively attached to the frame and selectively operable between:
a secured configuration to restrict movement of the coupler relative to the frame, and
a movable configuration to permit limited movement of the coupler relative to the frame about the coupler point.
XXVI. The mount assembly as set forth in clause XXV, wherein the perch of the coupler defines a perch pivot surface disposed in sliding contact with the coupler seat of the frame to facilitate selective pivoting movement about the coupler point when the coupler lock operates in the movable configuration.
XXVII. The mount assembly as set forth in clause XXVI, wherein the perch pivot surface has a generally spherical profile.
XXVIII. The mount assembly as set forth in any of clauses XXVI-XXVII, wherein the coupler lock includes a coupler retainer supported by the frame and arranged to abut the perch of the coupler when the coupler lock operates in the secured configuration.
XXIX. The mount assembly as set forth in clause XXVIII, wherein the coupler retainer includes a coupler end portion arranged adjacent to the perch, a coupler interface arranged for engagement by a user to operate the coupler lock between the secured configuration and the movable configuration, and a coupler retainer body extending between the coupler end portion and the coupler interface.
XXX. The mount assembly as set forth in clause XXIX, wherein the frame further defines a coupler retainer aperture extending in communication with the coupler scat and arranged to receive the coupler retainer body.
XXXI. The mount assembly as set forth in clause XXX, wherein at least a portion of the coupler retainer aperture is disposed in threaded engagement with at least a portion of the coupler retainer body such that rotational torque applied to the coupler interface in one direction urges the coupler end portion into abutment with the perch of the coupler to operate the coupler lock in the secured configuration, and such that rotational torque applied to the coupler interface in an opposite direction urges the coupler end portion out of abutment with the perch to operate the coupler lock in the movable configuration.
XXXII. A mount assembly for use with a navigable tracker, the mount assembly comprising:
a frame defining a coupler seat;
a coupler including a perch disposed in the coupler seat and arranged for selective movement relative to the frame about a coupler point, and a tracker interface spaced from the perch for releasably securing the tracker;
a coupler lock operatively attached to the frame and selectively operable between:
   a secured configuration to restrict movement of the coupler relative to the frame, and
   a movable configuration to permit limited movement of the coupler relative to the frame about the coupler point;
an anchor extending along an axis between a distal end for engaging tissue and a proximal end arranged to receive impaction force, the anchor including:
   a shank disposed along the axis between the distal end and the proximal end, and
   an arrow body coupled to the shank and having a tip tapering towards the distal end for advancing into engagement with tissue with a pair of wing braces extending away from the axis to inhibit rotation of the anchor about the axis relative to engaged tissue;
a guide operatively attached to the frame and defining a bore shaped to receive the shank of the anchor; and
a guide lock operable between:
   a released configuration to permit movement of the shank along the bore, and
   a locked configuration to restrict movement of the shank along the bore to effect concurrent movement of the tracker with the tissue engaged by the anchor.

1. A mount assembly for use with a navigable tracker, the mount assembly comprising:
a frame;
a coupler operatively attached to the frame for releasably securing the tracker;
an anchor extending along an axis between a distal end for engaging tissue and a proximal end arranged to receive impaction force, the anchor including:
a shank disposed along the axis between the distal end and the proximal end, and
an arrow body coupled to the shank and having a tip tapering towards the distal end for advancing into engagement with tissue with a pair of wing braces extending away from the axis to inhibit rotation of the anchor about the axis relative to engaged tissue;
a guide operatively attached to the frame and defining a bore shaped to receive the shank of the anchor; and
a guide lock operable between:
a released configuration to permit movement of the shank along the bore, and
a locked configuration to restrict movement of the shank along the bore to effect concurrent movement of the tracker with the tissue engaged by the anchor.

2. The mount assembly as set forth in claim 1, wherein the shank defines a shank diameter; and
wherein the pair of wing braces extend away from the axis to respective wing brace ends spaced from each other at a wing brace distance larger than the shank diameter.

3. The mount assembly as set forth in claim 1, wherein the pair of wing braces are further defined as a first pair of wing braces; and
wherein the arrow body further includes a second pair of wing braces interposed radially between the first pair of wing braces and extending away from each other transverse to the axis to inhibit rotation of the anchor about the axis relative to engaged tissue.

4. The mount assembly as set forth in claim 3, wherein the first pair of wing braces extend transverse to the axis to respective first wing brace ends spaced from each other at a first wing brace distance; and
wherein the second pair of wing braces extend transverse to the axis to respective second wing brace ends spaced from each other at a second wing brace distance.

5. The mount assembly as set forth in claim 4, wherein the first wing brace distance is larger than the second wing brace distance.

6. The mount assembly as set forth in claim 4, wherein the first pair of wing brace ends are further defined as a pair of wing brace tips; and
wherein the second pair of wing brace ends are further defined as a pair of wing brace edges; and
wherein the arrow body further includes a pair of tip transition surfaces extending between the tip and the pair of wing brace tips, and a pair of edge transition surfaces extending between the tip and the pair of wing brace edges.

7. (canceled)

8. The mount assembly as set forth in claim 6, wherein the arrow body further includes a pair of tip apexes defined between the pair of tip transition surfaces and the pair of wing brace tips, and a pair of edge apexes defined between the pair of edge transition surfaces and the pair of wing brace edges; and
wherein the pair of edge apexes are spaced longitudinally along the axis between the pair of tip apexes and the proximal end.

9. The mount assembly as set forth in claim 4, wherein the first wing brace distance is substantially equivalent to the second wing brace distance.

10. The mount assembly as set forth in claim 4, wherein the first pair of wing brace ends are further defined as a first pair of wing brace tips; and
wherein the second pair of wing brace ends are further defined as a second pair of wing brace tips; and
wherein the arrow body further includes a first pair of tip transition surfaces extending between the tip and the first pair of wing brace tips, and a second pair of tip transition surfaces extending between the tip and the second pair of wing brace tips.

11. (canceled)

12. The mount assembly as set forth in claim 10, wherein the arrow body further includes a first pair of tip apexes defined between the first pair of transition surfaces and the first pair of wing brace tips, and a second pair of tip apexes defined between the second pair of transition surfaces and the second pair of wing brace tips; and
wherein the first pair of tip apexes and the second pair of tip apexes are each spaced longitudinally along the axis between the shank and the distal end.

13. The mount assembly as set forth in claim 1, wherein the frame has an outer frame surface arranged adjacent to the bore, with a frame slot extending through the outer frame surface and into the bore to define first and second bore edges.

14. The mount assembly as set forth in claim 13, wherein the first and second bore edges are spaced from each other at a first bore edge distance when the guide lock operates in the locked configuration, and at a second bore edge distance when the guide lock operates in the released configuration.

15. The mount assembly as set forth in claim 13, wherein the frame slot defines first and second flexure portions; and
wherein the guide lock includes a guide retainer operatively attached to the frame to urge the first and second flexure portions towards each other in response to changing operation from the released configuration to the locked configuration.

16. The mount assembly as set forth in claim 15, wherein the first and second flexure portions of the frame are arranged to resiliently move away from each other in response to changing operation from the locked configuration to the released configuration.

17. The mount assembly as set forth in claim 15, wherein the guide retainer includes a retention portion, a guide interface arranged for engagement by a user to operate the guide lock between the locked configuration and the released configuration, and a guide retainer body extending between the retention portion and the guide interface;
wherein the frame further defines a guide retainer aperture formed extending through the first and second flexure portions and arranged to receive the guide retainer body; and
wherein at least a portion of the guide retainer aperture is disposed in threaded engagement with at least a portion of the retention portion such that rotational torque applied to the guide interface in one direction urges the first and second flexure portions towards each other to operate the guide lock in the locked configuration, and such that rotational torque applied to the guide interface in an opposite direction permits movement of the first and second flexure portion away from each other to operate the guide lock in the released configuration.

18. (canceled)

19. (canceled)

20. The mount assembly as set forth in claim 1, wherein the proximal end of the shank is shaped to enter into and pass through the bore of the frame to bring the shank into sliding engagement with the bore when the guide lock operates in the released configuration;
wherein the anchor includes a head coupled to the shank and arranged at the proximal end for receiving impact force to advance the arrow body into engagement with tissue; and
wherein the shank of the anchor has a generally cylindrical profile disposed in engagement with the bore of the guide such that rotational torque applied to the anchor effects rotation of the shank relative to the frame when the guide lock operates in the released configuration.

21. (canceled)

22. (canceled)

23. The mount assembly as set forth in claim 1, wherein the coupler includes a perch arranged for selective movement relative to the frame, and a tracker interface spaced from the perch for releasably securing the tracker;
wherein the frame further defines a coupler seat supporting the perch for selective movement relative to the frame about a coupler point; and
further comprising a coupler lock operatively attached to the frame and selectively operable between:
a secured configuration to restrict movement of the coupler relative to the frame, and
a movable configuration to permit limited movement of the coupler relative to the frame about the coupler point.

24. (canceled)

25. (canceled)

26. The mount assembly as set forth in claim 23 claim 25, wherein the perch of the coupler defines a perch pivot surface disposed in sliding contact with the coupler seat of the frame to facilitate selective pivoting movement about the coupler point when the coupler lock operates in the movable configuration.

27. The mount assembly as set forth in claim 26, wherein the perch pivot surface has a generally spherical profile.

28. The mount assembly as set forth in claim 26, wherein the coupler lock includes a coupler retainer supported by the frame and arranged to abut the perch of the coupler when the coupler lock operates in the secured configuration;
wherein the coupler retainer includes a coupler end portion arranged adjacent to the perch, a coupler interface arranged for engagement by a user to operate the coupler lock between the secured configuration and the movable configuration, and a coupler retainer body extending between the coupler end portion and the coupler interface;
wherein the frame further defines a coupler retainer aperture extending in communication with the coupler seat and arranged to receive the coupler retainer body; and
wherein at least a portion of the coupler retainer aperture is disposed in threaded engagement with at least a portion of the coupler retainer body such that rotational torque applied to the coupler interface in one direction urges the coupler end portion into abutment with the perch of the coupler to operate the coupler lock in the secured configuration, and such that rotational torque applied to the coupler interface in an opposite direction urges the coupler end portion out of abutment with the perch to operate the coupler lock in the movable configuration.

29. (canceled)

30. (canceled)

31. (canceled)

32. (canceled)
